SEQUENCE LISTING (71 sequences)

SEQ ID NO: 1 - DNA, 13782 bp, maize fine streak virus
  CDS (254)..(1642): N protein
  CDS (1946)..(2962): P protein
  CDS (3165)..(3446)
  CDS (3532)..(4515)
  CDS (4744)..(5484): M protein
  CDS (5733)..(7523): G protein
  CDS (7664)..(13498): L protein
acacaccaaaaagggtgacgcatccaagaaccggaaattgagtcattgctggatcctccatatggtggattcgggcaccgtcctgggccccgctgtagtcgtgctggctagcggtcacacaaaacgagtgtccaaaactcagaaacaaatcgagacaatccgtcatccgattatttaaataaaacatcaacctaaagccacctcattctccaaccaacaacacaacacatcactccaaaactcccacacaatcatgaattacaaccgtttgaaattcgaggatgcattcctcacaccaagtgctgactatgctgcctttaaaacccctcttccatatcctgacctcaccacaggacccacagaagtcccctacactaaagagaagcttataactgcgagttatccttactggactatgacaaaagtcaactcaggggacatcaagacactaggaaagacagtattgacaaagttatctgataaaaagatcactgaaaagaccttgttcgacatgtgcagaattgccctatccttgaaagcacctaatggccaagacgtgttcaagataccaacagtcacatttgcaggagccaaaacaggagggcaggacgatactaacctggtaccagtgccttctgacacttcaaaaacaactgaatatttacaaacagtcaccagcaagagttcaacctcccaggctacccaaaagaaatcactgtctacctatgttgataatgacgccaattatgaaactgccatcccctatttcctctgctcattcttacgattgatagtgaaacaacctgagagctggagaagagcatttggagatatcaaagaacaatacggtaagttttacggcaaaaccagcaacctgataactaacgcagaagatgatctatcaatcgcaagtgtggtcaaagttgcatttgatacatttaaaccaattgtcaactttatagcttatgtggcaggggagacagacaaaggactttcaccaaacaccaaggaacatggaatgtttacatattttgttggtcaacatctgtcctttgtaggaatgcatgtctatcctatggtggcagaactaatgcagaaatgtaaagggattaaacaggacctattcctgacattcttagatgttgatgaaacaaaagagtctgtaaaagaaatttgcagaatcatgacaacatatgatgcaccaagtcaaactgatagggcaagcagagatttcctgtggaaatatgccagagttatagatgaagggtttttcttgaagcttcaaaataagaaaaataaagagttcttgtatggccttgcactaattcatgagaaaatgggactagtcagaagcgtgtcatatgccaaacccaccaacatggcaatactacagaatcaagatggattaagagcagatgcagaggaatatgccaatctgtttgtcttcctgtacaaaaaggcaacaggtcaaaaagaaggagcatctcctatggatcaactgagatcacaattggcaggggagagggtcacagcaacaatagcagaagcatccgcgccaaagagatcgtccgacggcactgggaacgtgagcaagaagaagtccaggaaggacgatacaggaattgcctcgatggaattttaactaagtgccattaatgttaccttattgttcctatatctatgttaagaagtattataatagtattatatgatataatgtaatgtttctattgtgattgtgaggtacaaataactagtaatctggaccttctacgataaatgttgagagtaatataagtgcaatgtcatttcttatatggggatatgaatattctctttggattagagtgaatcctatatatattttcgatatcctatttacaaagagtaatgtaataaggttgtgatgagtatttaaataaaacatcaacgttcagatgacaggatgagtcaaagaactctgcgaagcaccagatcttcacaatcaaagaaaggatttgcaaacctagagtcctatagggaaaagtcagtgacagaagcagacccagcatattcatctgtacaaatcccacactctgtgacagaatccacgcgttacaaatctcttgaacaagtagataaacttcctctttcatctgatatcccagcccaaatcgaaagtcaggtagagatggctccgattctaactcctgagaggaaggaaaccatgaaggaagagatcactaaatttttgatggctagaggatgtgttccggacaacatggcaatggagcaactactcaaggctcatgaacccataatagacaatccagctgctgacattatacctgacactacctatttgtccgcattttgtcagggatggacattctgcatgagagtatatagtgacatggctctcaataatctgaaaaagaccatagaacctatcatgtccactcttgcagcaacaacaaacactatgcaagggttaacaggagagctaaaccatacatcaaaactcttgaaactaagggtatcagctgtgcccttcaaaaggcaaagccccaaggatgtagcagctcgcatcaacaagatgaaacaaaagggcaggagagcagaagacatcatggatccgatgaagcaaccaatggtggaagaggaggaagatatagatgaagagtctgaggatgtctttcctgaatacagctccagtctcctgaggacaacatgggatgagatgagtgatgacctgaaaaggagtctcctatacgggattattaaagctgcaatggatattgaagatgtgggcgaacttgactcagacacaatgaacaccatagagcggtcactaaaaggaagccaactcaccacagcatatctaatgaaggatgaaggagtggatcgcgacaaaatcaggaggtatatcaagaagatatgggaacaagctgggctgcctaacaagatggagattggggactaaatcatataataaataaatgtatttatggtgatgattaattatcgtgtccatcactccactcgattcaccggttcacagtgaaaatgcatccccacattgtactacctatgtcctacttaattatgtatcttaaaatatatagtgtgctattatgtaatcatgccaatcagttatcccattatataaataaaacatcaacataatggattcaggaggagaaatcactcaaacacccaccagagaccgtcacgtgagaataagacatgagatcaactctcctataggatcagatttcagcatcagtacaaggaaggacagagaagttgtatttcaactgacggaccaaggcaacacatggttcagaggtgagagccattattgggtaggtaatgaacatctccctattattgtgacatatgattcgaaaggtggagatacaataagtgatctaattgtagaggtggagatgaaattaagattctgatgattggttatatgttattgaatctgcatccccgctgtaattggtgtatgagtatctatttaaataaaacatcaatatactaaagatggattccaggtcgtttgtagacaggactcccaaccagaaagcagacagaaaagcactgacaaaagcatccaaagcactgttcaaaggaaagataaaatctgaagaattaggaattattagaaaggcttatgcacatacaatctcaattacagacattatcagaggacagacatctgagggaggagagaaatctctattacaaattgaaggattggtattcatgtaccagccagaggtttcaagttttgttaatgaaggagtggagataagcatagtatcagaggagataccaggagagatagaaccactatgttccttcttgttcagagcatctgattatgttaaatctgtcttatcactccaatggacattgaggacaagagaatttagcaatccagacaaaaaccccctatatatcacttacaggacattaaacaatgtacacaacgtctctttaggaagggtgatcggaggagtcattggacactggaaagaaaacaaagatggggcaagaactcataaatttacaaccccatcgggttacactaaggtctatcctggtgagtatgcattggacattagtcatatgaaatactatcatgccggaggatcatatatagttcagagctcatcggaccttcggaactttgtgaagttcaaacaagccatgtcacagtatataggagatcatgacaatgatgtgtacttcgatatattatcacgaagtccactggtgatcagagaggctatacagagaatcatggatgaaaaacccaaaccggatgactactatcatataatgacttatgtcaagcccatgagatccaatgtccacgttccatccatcggataccgtgatctaatttcattcaccaaggaggaagtctcatcactcacatacttcaatacaggtgatgagatagaagaaccacagcatctggagacaaaggtatcatccaaagccgcagtaccggctttctaatcagcttgagttggagagtgcatatcggggtcagtcatacgtgttgagatcccactatcccatgaagagttctgaataattaattatataaataaagaactctttaccaatagacgctagtgggagaacccctgcctatgtgtattaaaagcgtgtctaaatgtattattgtatgattatttaaataaaacatcaacagcaaagcttccaactagtaagcccagcataatggcaatccatcctgcacagcttcaaccctatatgatcacttattattacaagttcgggatcactaccacagaaagtatccctatcaacgagcaaaaatttgtatcgtatgctattaaaaccgctatattagcattaaatgatgatgacccaaggctggtagaactaaagaaagtaggtgattttgatgtatcaagccgattgttatttattcagcaccatggaagatctcttgaccatcctttagtgtcaatggacaagccgatagtaaagcttgagggggccctaagtcttgaatatctaactgcgggaggaacttatcagccatacaatacacatagagcattagtcagaatgagatcaggcaaagaaggattattacaaatgaaatggcaatgggcagatgaaaatgacgaagatagtacatatgtatctggggtaatatatagtgatctagtgaacatcactgaagaagaatatgaaacttgtatgcagaataatattccaacattcgacctgcttggagctcttcgagagttcaatctcaagaacaagaaaaatagggaatcacaatatgacgacaagaaagaagacaaagcagagaaggcgacaacagagaaaaggaaaaggcaagaatcaaaaccttctctctatcaatccttcaagtcagtattcaggccacaccacgatgatgaggatgataaaccaggaccatcaaaacaattaactctaaggatgtgaagttcatttacctataataaattatgaggaatagtgttctcacctactcatatattcatgatcttgtaaaataatatagatatcgaccccacatccctatgacatctcccgtctcccctctccgtcacaccgccatccaccagtgcgggtcagcggcctagaagaccaggatgtatttaaataaaacatcaactcttgaaggcaccacacccacaacaccacacagcacaacacgacagccagagacaatgatggcaagactggttccatgcttcaccttagcactgttactacacttaacagaatgtgctgactatatcacatcaccaattaggcccatgtatgaatgcaactcatttgattcctcagtcacattatcgtcctggtaccaaacatgcagggcaagctgtagacctgatgcaggctgggcaaggtcaaatatcacattatattatgacacatttgcagggagtgtaatacctgtatatcaaatgcagatttatagaaccaccatagaatctcacacggacatattggacaattgcaaatatactgaatattcaactcccattatcatagataaagatgatataactatgtacactacaaaattatttactactgccacgctcaacccgaacaactcgacactgataatcgaaaacaagaccatcccagaatgtagttacttcagggacaagtattcttcatatgacaccatcattgccactaagtacatggcgcgggtaatgagcagctcagtggcttctgaatattacttgaaaataccagcattgcaggcaacaatgagttacagttcaggaagcgccagatatgggtcaggagtgctagtatggaatataacattaccgaacataaatcagtgcaccctttctgatgggattacagagacttgccggagtgactctatgttaggaaaatggtggtgtccttccctgattggatcaatattgcttactaagggagcaacagccacttgtaaaggtgacctaattcaagtgtctccgggaatttatgtaaccttatctggcacatcaacagtagacagtacagccagtatgctccaggatggaattgatgaacttgaaccagatcttgtatcagttctaacacagatcaacaaggctatgactacctttgcatcagcaagttgtgagagcatttgtgatctggcggatcagaccttttctctaagagaaaatacaacgtcagttgtggaaacacccattggaccatggcttggatggcgagagtcattatcaaacttaaatcatatttattatgtgtcagcatgtcgacgagtaacccaatggaagatcatgtcacctttaaatccgtgtgacagtacggtcgggttaacaattacagacggggagaaggggctttcactgtgggatccggctcaaaattattttattatgggtgcagaatgtgcggactttgttcgctctgattggaaaactctgatgggcaagaagcaatctatccagatccacttctggggaaagaaattgattatctatccaccatatattgccaatacatcttgggaggaatcagaattgcatatacatagaacatcaaagtggacaccctatattgatggacaagacataaatacagaagatgatatgctgactctacttgatcaaatagaacatcactcaaggaaattagtgagggtcataggaatggaacagaccaagaacttgaccattttcacaaaattatggactggaatatcagatattggatccactatcagtggcatataccatggtgttgcatctgcgataggacatttcttgaatttcatcataaagttggtcattggatttaccgttggaacaataatgttatatatatcatggatcataatcaggaagctcatattaaaacagaaaaacagaaagccagagcaagaccaggaaatgatggatgtggctccgatacacaggtccgggggtctgacacagcctatattgaggagaaaaggaatatctatggatgacatgtgatgaaggtccatcgagcagaaaataagagcccttacaatatgtgtcccactacaatgaacaatgtagttgagtggcggaaggaacagtgtatgatatgtagtactcgaataattaattatttaaataaaacatcaaccatcatggatccagaaatctatgattcaattctgcaagattgggaaaatgaaggagactcagctgttgaaagaactccatcagtgagacttaagtcagcattgaagactcttcccgattatttgaagaataagacagcaaagagaagatatgaggctgtgacagaggtagtcagaccgaaggcccttgacgactcaaccatagtgttgggcaagatatgggacaaggcaaagtctgaacatgtcgggaagttctatcacttcgttgaatccactttcccctcatatgaccgaggccttgttgagattttccgagcagaaatccagatcctatgtggaaatacacaagggagtaactgtgatgtcactttgaactcagtaatatacggagaattgaaaaatatagcacccaatttgctccctctctatctgtttttctcttttcttgtgactatctcatgggggacatacaccaacacaacaaggtatgcttggccagtcataaaagagtctatcggaattttatgggctgaatatgatgagaaattcaaggttggaatatacgatatgtgggcactgttacaagcaaaatatcatgcacctataatcatccatcgggatcatctgctcaatataactgataagttgggggagcgattgaattgttttatattctctagtatatcaaaccaagttcaagatatgctctctgtgtcaatgaattgtcttgacagagtgatggcatggggagatactgtcatcaaaaaccacggttctgcagggttccagttattatcgaattatgaaccacttgttgtcggagtattactatctagaggggatgacaggctatgggatacaaccagattcttaacgaatgtgattagtgatattaaagaagagctctcatgggcagtgttctatgttgatgagctattagaactattatctggattatccatagatcaactgtgcgacattcatggactatacaggatttgggggcatcctgaggtacaactaaagcagggactagaaaagatgttcaaaatctcaatggctgataaggtcatagatcaagaaaatattaatatatctggaagaatgttcaaagaattgttcttcaggaattacaagaagatccacaagcattatcccaattttactttcaacgtaattgatgaaagcgagttgaatgagttactcttgacagattatgtaatgaaatgtctcaatgataatcaagagataagaattgatgaggcaagatatgaattaaattcatgggacagagtctcaattttgaaaaattttgagattccgtattcatggaacttggcgcacaatataaaagacaaggctgtgtctccagacagagaagaactaaaaacatctgtcagaaggggattgggagccatatggcaagatgctcggagaactgtattaaaatggttgaatctcaacatcctctctatgagggattttttatccaacattgatcgattcggaatggatgttaaacacttgattattggtctatatgccaaagaacgagagttaaaatattacccaaggttcttctccttgatgtcattttatctgcgattatatgttgtgtcaactgagcagttgatatcagataaccttttacagtacttccctcagataacaatgacatctgacctattatctatgaccaagaagatactgaaacttagtggtaaaatgaaggagaaagacgatgacttcaacacaaatgaaacaaggaagaaggaagtttcatattctattaacattgactttaaaaagtggaatcaacagatgagatatgagataacagaacctgtattcactcagatagatgccacatttggtttcaccaatgttattcgaaggacccatcaattcttccaagggtgttatgtgtatctttgctcaggagaatataccccagaaatttcaggcgatagcttcatactagaacctccctggtcttggaccaatgacggatctggaaaagagggtcttagacagaagggatggaccataatgactgtctgtatgattgagtatgtgatgagacaacacgcagcacggtcacagctgatagggggcggggacaaccaagtacttgttgtgacattggagacagaatcattaaacaatgacgggaccataacaaatgagggaaagagagagttaaaggaaaggatggaaaagatactgaaagatctcgactctttcttcacatcggtcgggctgcctcttaaaataaatgagacctgggtaagctctgagttatttatgtacaacaagatcatgtgttacagaggaagaacattgagaagctgtctcaagactgcatcaaggtcgttccctttctccgatgatgcagccacaactctacatggaatcatgtcaacattgggcaccagcgtcaagtcactgtgttccaaagacttctctcaccttccagcatatgtgttctcaagatggcttgtactaattgcttcatacctagtcttttactttcacccgttgatcccagggggcatcaacaacctgccgtcaattcagtctcgttatggaaatatggggagctttgtgaagatgcaagtccagcgatcaaatatgactatcaagaaactcattactcgatgtttattctatttcagaatattcggaggatatggtattggtgtgccgagtgactgggttatgagaggattcccagatccgctgacaagtgcaatagcctggattaatagatgtctttctttaatctcagggaccaacccttacagggacgaattgacaagtttcatatctatgagcacaaagcctggagatccctcatacctgcaccttattagtgatcctgtatcaatcaaccatgatggcccgatccatgggttggccaggttaagggaagaggcagaaaaggcaattgtagaatcacctctcgtcaaaaaccataatttgaaagaaattgcaaaagtatgttactcccaaaattcacaatctcttctgaacgccatgtgctcatcaaatgtactagaacctaaattcctccacgatatatttagcgctacattgtatggatatttcaatagcattgttagcagggtggataaatctgctacaatcaccagattaaacaagaaggcaaacgtgatggaagcattacgcatcactgaaattgaatatatcctatactcactagaaaggagttctcaccactacgacatgaaaccagcaacatgctctaggattactgcagagatatatcgctctacatcatggaaaaaaccaatagtgggagtcacagtcccaagtccagtggaagtgttttcaacgataagagaatcatggcatactgaatgtgatgaaaattatgttttgttgaatgttatggagaagcccaataaagaaagaccatacggaccactggaaccttacttagggtcctttacagatgagaagtttaaattatcccctctggcaaatgcatttggagaagagtctctgttgaacaaagcaatccatatacagaaactgagatattggagatataataaaggaactgacctcttcaatcttatcgaaacaatactaaaaagcgaatgtgacttagatcctgatagtgtgttcatcctccattcatcatccagctctgccacacatagatactcaggagaggtgttagctcacggtgccataatgaatgcaaatccggacgcttatcgctattttgcaatgacaaccagcactctacaaaagaggtcaaagggaggacggaacgacatgattcactttcaggggagtctgttgtggatctccagcatgatggtgaataaagcaacaatagcagagatagagagtgggacatatcacttccatgaggaatgtgacacttgcatttatcctctatatgaagtgttggatgaagacactacactcaatacagttcctaatgtgccgattatgaaagacaacacacttgcctatgtaccttcaaacatgattcagatatcatatgctgatgaagtgctaatcaagaaataccgatcacttagatgtcagattgcagaccctcaagttgcagtcaaggaggagctaatatatgatgctatatcacaagtgctctttgtacttttaacaggaaagagagtattgacagggtcacaagtaaacaagctgttcagcaagataaaacctgatatggtatttgggaaactcaaaacactcttgaatttgcatgaggagtttctaaagaatcggggtacaatcaagtggggaaaactggatcccatcctcagtaatgtaaacatcattgacatgatcaagagactaggacttagtgtcccaagaaaacatctacacaaaaaaggaatctcatgtagcctattggtcccgcagattatgatgaaagtcaatcctgaagacttgggctatcttacaactcttgatatcgacccttggattattattgcagcgaggtgctatttaaaagaccctgctttgaggacctgtgaagaatgcaagaaatctctgttgagcctctttgtatccaatcttgagttctgtgatttggagtactcctgcgaatttcacactgcaaggaaaatcccccaactggtatttgtccctacagatgcctactcaatatcaaaagaaggtacatattctttatctaatggagggaatgagaagacaaccactagggtcactgcaaatgcactgaaggtgattcccagggtgaaggtgccaattcctattactctgcctccgtattcctgcatgagagagatcatctcaggatcggatatttgttctctggcatatatgatgttgtggttgatgtcgaaatccgaagcaaattctcagatcctaattctcacagaggcagactggaacatattattcatgctaatacatatagccaaagtgacaagaatagagatcaggattgtcaccaagctctctcagaactccactacaggatctgaattctcaaaccttgtgggatttagggatggcctaagagaagaggacaagaggttcataatcattgatgaaccacaagagcatactctgagagatggaaactgtatacaggtggttacctatcaaacggggcaggtatatgatacaaaatacattgcttttccatcaactagcatactgtgttttgggaaagtccaagggatgatactcacacggagagaattatattatccatcggtccaaattgacagatcgaactggtccattgcagagagatcaaccaatgaggggtgtgatatcacagttcttcataatatgaggcatcatttcatgaaaataaagaaagtcaatctacagaaagctctaattgaagaaatgttagattcatcgatctatcctatcagatcccataatgctatggccaggggcttacccgaaattgaacaatacaagctaccagtatcaagacttcagcttatctacaacgtacaagtattaatgaaacatctggacaatatgccgatgaagtggagaagaaccgtggagggggtttggaagtatcactgtcatgcattcatcataatgcttgcattaagtctgccacctttcaagagcggaaggttgctgaaggggttgcaggagtataggtatgatcaattgaccaagacattgatccttaagcggagaaatgtgtccaatgctcttctttgtgatacatttcaatatttcctggaagattgcggaagattcagagtgcccattaagatcaccgggtataacaaaatattccttgtggaggttgatgaatcactcaaggggattcatgttatttgatggaggtgggtggactgtggggtgcgtgctggttggtgctcgattgtgctgttggatgggggtggcgggggagaggggccgtgttgggctgttgactgttgttgggccccgagtacatgtcagatttaacaaaaacatctatttctcgacgacgcccaattgacgggtgttgtgtatggtgggactcgtagcacgacgctcccttttaccaggcctaactttgattgactttagggaaattcccccatcatccgagttgtgatgcgtcatttttttgcctgtgt

SEQ ID NO: 2 - protein, 462 aa, maize fine streak virus
MNYNRLKFEDAFLTPSADYAAFKTPLPYPDLTTGPTEVPYTKEKLITASYPYWTMTKVNSGDIKTLGKTVLTKLSDKKITEKTLFDMCRIALSLKAPNGQDVFKIPTVTFAGAKTGGQDDTNLVPVPSDTSKTTEYLQTVTSKSSTSQATQKKSLSTYVDNDANYETAIPYFLCSFLRLIVKQPESWRRAFGDIKEQYGKFYGKTSNLITNAEDDLSIASVVKVAFDTFKPIVNFIAYVAGETDKGLSPNTKEHGMFTYFVGQHLSFVGMHVYPMVAELMQKCKGIKQDLFLTFLDVDETKESVKEICRIMTTYDAPSQTDRASRDFLWKYARVIDEGFFLKLQNKKNKEFLYGLALIHEKMGLVRSVSYAKPTNMAILQNQDGLRADAEEYANLFVFLYKKATGQKEGASPMDQLRSQLAGERVTATIAEASAPKRSSDGTGNVSKKKSRKDDTGIASMEF

SEQ ID NO: 3 - protein, 338 aa, maize fine streak virus
MSQRTLRSTRSSQSKKGFANLESYREKSVTEADPAYSSVQIPHSVTESTRYKSLEQVDKLPLSSDIPAQIESQVEMAPILTPERKETMKEEITKFLMARGCVPDNMAMEQLLKAHEPIIDNPAADIIPDTTYLSAFCQGWTFCMRVYSDMALNNLKKTIEPIMSTLAATTNTMQGLTGELNHTSKLLKLRVSAVPFKRQSPKDVAARINKMKQKGRRAEDIMDPMKQPMVEEEEDIDEESEDVFPEYSSSLLRTTWDEMSDDLKRSLLYGIIKAAMDIEDVGELDSDTMNTIERSLKGSQLTTAYLMKDEGVDRDKIRRYIKKIWEQAGLPNKMEIGD

SEQ ID NO: 4 - protein, 93 aa, maize fine streak virus
MDSGGEITQTPTRDRHVRIRHEINSPIGSDFSISTRKDREVVFQLTDQGNTWFRGESHYWVGNEHLPIIVTYDSKGGDTISDLIVEVEMKLRF

SEQ ID NO: 5 - protein, 327 aa, maize fine streak virus
MDSRSFVDRTPNQKADRKALTKASKALFKGKIKSEELGIIRKAYAHTISITDIIRGQTSEGGEKSLLQIEGLVFMYQPEVSSFVNEGVEISIVSEEIPGEIEPLCSFLFRASDYVKSVLSLQWTLRTREFSNPDKNPLYITYRTLNNVHNVSLGRVIGGVIGHWKENKDGARTHKFTTPSGYTKVYPGEYALDISHMKYYHAGGSYIVQSSSDLRNFVKFKQAMSQYIGDHDNDVYFDILSRSPLVIREAIQRIMDEKPKPDDYYHIMTYVKPMRSNVHVPSIGYRDLISFTKEEVSSLTYFNTGDEIEEPQHLETKVSSKAAVPAF

SEQ ID NO: 6 - protein, 246 aa, maize fine streak virus
MAIHPAQLQPYMITYYYKFGITTTESIPINEQKFVSYAIKTAILALNDDDPRLVELKKVGDFDVSSRLLFIQHHGRSLDHPLVSMDKPIVKLEGALSLEYLTAGGTYQPYNTHRALVRMRSGKEGLLQMKWQWADENDEDSTYVSGVIYSDLVNITEEEYETCMQNNIPTFDLLGALREFNLKNKKNRESQYDDKKEDKAEKATTEKRKRQESKPSLYQSFKSVFRPHHDDEDDKPGPSKQLTLRM

SEQ ID NO: 7 - protein, 596 aa, maize fine streak virus
MMARLVPCFTLALLLHLTECADYITSPIRPMYECNSFDSSVTLSSWYQTCRASCRPDAGWARSNITLYYDTFAGSVIPVYQMQIYRTTIESHTDILDNCKYTEYSTPIIIDKDDITMYTTKLFTTATLNPNNSTLIIENKTIPECSYFRDKYSSYDTIIATKYMARVMSSSVASEYYLKIPALQATMSYSSGSARYGSGVLVWNITLPNINQCTLSDGITETCRSDSMLGKWWCPSLIGSILLTKGATATCKGDLIQVSPGIYVTLSGTSTVDSTASMLQDGIDELEPDLVSVLTQINKAMTTFASASCESICDLADQTFSLRENTTSVVETPIGPWLGWRESLSNLNHIYYVSACRRVTQWKIMSPLNPCDSTVGLTITDGEKGLSLWDPAQNYFIMGAECADFVRSDWKTLMGKKQSIQIHFWGKKLIIYPPYIANTSWEESELHIHRTSKWTPYIDGQDINTEDDMLTLLDQIEHHSRKLVRVIGMEQTKNLTIFTKLWTGISDIGSTISGIYHGVASAIGHFLNFIIKLVIGFTVGTIMLYISWIIIRKLILKQKNRKPEQDQEMMDVAPIHRSGGLTQPILRRKGISMDDM

SEQ ID NO: 8 - protein, 1944 aa, maize fine streak virus
MDPEIYDSILQDWENEGDSAVERTPSVRLKSALKTLPDYLKNKTAKRRYEAVTEVVRPKALDDSTIVLGKIWDKAKSEHVGKFYHFVESTFPSYDRGLVEIFRAEIQILCGNTQGSNCDVTLNSVIYGELKNIAPNLLPLYLFFSFLVTISWGTYTNTTRYAWPVIKESIGILWAEYDEKFKVGIYDMWALLQAKYHAPIIIHRDHLLNITDKLGERLNCFIFSSISNQVQDMLSVSMNCLDRVMAWGDTVIKNHGSAGFQLLSNYEPLVVGVLLSRGDDRLWDTTRFLTNVISDIKEELSWAVFYVDELLELLSGLSIDQLCDIHGLYRIWGHPEVQLKQGLEKMFKISMADKVIDQENINISGRMFKELFFRNYKKIHKHYPNFTFNVIDESELNELLLTDYVMKCLNDNQEIRIDEARYELNSWDRVSILKNFEIPYSWNLAHNIKDKAVSPDREELKTSVRRGLGAIWQDARRTVLKWLNLNILSMRDFLSNIDRFGMDVKHLIIGLYAKERELKYYPRFFSLMSFYLRLYVVSTEQLISDNLLQYFPQITMTSDLLSMTKKILKLSGKMKEKDDDFNTNETRKKEVSYSINIDFKKWNQQMRYEITEPVFTQIDATFGFTNVIRRTHQFFQGCYVYLCSGEYTPEISGDSFILEPPWSWTNDGSGKEGLRQKGWTIMTVCMIEYVMRQHAARSQLIGGGDNQVLVVTLETESLNNDGTITNEGKRELKERMEKILKDLDSFFTSVGLPLKINETWVSSELFMYNKIMCYRGRTLRSCLKTASRSFPFSDDAATTLHGIMSTLGTSVKSLCSKDFSHLPAYVFSRWLVLIASYLVFYFHPLIPGGINNLPSIQSRYGNMGSFVKMQVQRSNMTIKKLITRCLFYFRIFGGYGIGVPSDWVMRGFPDPLTSAIAWINRCLSLISGTNPYRDELTSFISMSTKPGDPSYLHLISDPVSINHDGPIHGLARLREEAEKAIVESPLVKNHNLKEIAKVCYSQNSQSLLNAMCSSNVLEPKFLHDIFSATLYGYFNSIVSRVDKSATITRLNKKANVMEALRITEIEYILYSLERSSHHYDMKPATCSRITAEIYRSTSWKKPIVGVTVPSPVEVFSTIRESWHTECDENYVLLNVMEKPNKERPYGPLEPYLGSFTDEKFKLSPLANAFGEESLLNKAIHIQKLRYWRYNKGTDLFNLIETILKSECDLDPDSVFILHSSSSSATHRYSGEVLAHGAIMNANPDAYRYFAMTTSTLQKRSKGGRNDMIHFQGSLLWISSMMVNKATIAEIESGTYHFHEECDTCIYPLYEVLDEDTTLNTVPNVPIMKDNTLAYVPSNMIQISYADEVLIKKYRSLRCQIADPQVAVKEELIYDAISQVLFVLLTGKRVLTGSQVNKLFSKIKPDMVFGKLKTLLNLHEEFLKNRGTIKWGKLDPILSNVNIIDMIKRLGLSVPRKHLHKKGISCSLLVPQIMMKVNPEDLGYLTTLDIDPWIIIAARCYLKDPALRTCEECKKSLLSLFVSNLEFCDLEYSCEFHTARKIPQLVFVPTDAYSISKEGTYSLSNGGNEKTTTRVTANALKVIPRVKVPIPITLPPYSCMREIISGSDICSLAYMMLWLMSKSEANSQILILTEADWNILFMLIHIAKVTRIEIRIVTKLSQNSTTGSEFSNLVGFRDGLREEDKRFIIIDEPQEHTLRDGNCIQVVTYQTGQVYDTKYIAFPSTSILCFGKVQGMILTRRELYYPSVQIDRSNWSIAERSTNEGCDITVLHNMRHHFMKIKKVNLQKALIEEMLDSSIYPIRSHNAMARGLPEIEQYKLPVSRLQLIYNVQVLMKHLDNMPMKWRRTVEGVWKYHCHAFIIMLALSLPPFKSGRLLKGLQEYRYDQLTKTLILKRRNVSNALLCDTFQYFLEDCGRFRVPIKITGYNKIFLVEVDESLKGIHVI

SEQ ID NO: 9 - RNA, 15 nt, maize fine streak virus
  misc_feature (1)..(15): 3′ to 5′ gene junction sequence
uuuauuuuguaguug

SEQ ID NO: 10 - RNA, 15 nt, maize fine streak virus
  misc_feature (1)..(15): 3′ to 5′ gene junction sequence
uuuauuuuguaguua

SEQ ID NO: 11 - RNA, 15 nt, maize fine streak virus
  misc_feature (1)..(15): 3′ to 5′ gene junction sequence
uuguuuuuguagaua

SEQ ID NO: 12 - RNA, 30 nt, maize fine streak virus
ugugugguuuuucccacugcguagguucuu

SEQ ID NO: 13 - RNA, 30 nt, maize fine streak virus
acacaggcaaaaaaaugacgcaucacaacu

SEQ ID NO: 14 - RNA, 30 nt, Sonchus yellow net virus
ucucugucuuugagucuuuuauguuagugg

SEQ ID NO: 15 - RNA, 30 nt, Sonchus yellow net virus
agagacaaaagcucagaacaaucccuauac

SEQ ID NO: 16 - RNA, 30 nt, Rice yellow stunt virus
ugugguggucuauguaagacauuuaucaaa

SEQ ID NO: 17 - RNA, 30 nt, Rice yellow stunt virus
acaccaccauauccaaagccgccaugugug

SEQ ID NO: 18 - RNA, 30 nt, Northern cereal mosaic virus
gugcuggucacuagcuuguuggacuuagua

SEQ ID NO: 19 - RNA, 30 nt, Northern cereal mosaic virus
acgaucaagugagcggaccugguaagcauc

SEQ ID NO: 20 - RNA, 32 nt, Lettuce necrotic yellows virus
aaugccuguuauuaucuucuuuuuuuaguuca

SEQ ID NO: 21 - RNA, 30 nt, Lettuce necrotic yellows virus
acggacgauaauaaaaucaaaaaguccaau

SEQ ID NO: 22 - RNA, 14 nt, Sonchus yellow net virus
auucuuuuugguug

SEQ ID NO: 23 - RNA, 15 nt, Rice yellow stunt virus
auuauuuuuggguug

SEQ ID NO: 24 - RNA, 16 nt, Northern cereal mosaic virus
auucuuuuugacucua

SEQ ID NO: 25 - RNA, 13 nt, Lettuce necrotic yellows virus
  misc_feature (10)..(10): n can be a, c, u, or g
auucuuuugncuu

SEQ ID NO: 26 - RNA, 14 nt, Vesicular stomatitis virus
acuuuuuuuguuug

SEQ ID NO: 27 - RNA, 14 nt, Rabies virus
  misc_feature (11)..(11): n can be a, c, u, or g
acuuuuuuugnuug

SEQ ID NO: 28 - RNA, 24 nt, maize fine streak virus
guaguugagaacuuccguggugug

SEQ ID NO: 29 - RNA, 21 nt, maize fine streak virus
aaacucuugaaggcaccacac

SEQ ID NO: 30 - RNA, 24 nt, maize fine streak virus
guaguugguaguaccuaggucuuu

SEQ ID NO: 31 - RNA, 21 nt, maize fine streak virus
aaaccaucauggauccagaaa

SEQ ID NO: 32 - RNA, 21 nt, maize fine streak virus
aagccaucauggauccagaaa

SEQ ID NO: 33 - RNA, 21 nt, maize fine streak virus
aacccaucauggauccagaaa

SEQ ID NO: 34 - DNA, 16 bp, Artificial
  synthetic oligonucleotide
aaatctctgttgagcc

SEQ ID NO: 35 - DNA, 22 bp, Artificial
  synthetic oligonucleotide
ggtccattgcagagagatcaac

SEQ ID NO: 36 - DNA, 24 bp, Artificial
  synthetic oligonucleotide
ctatcctatcagatcccataatgc

SEQ ID NO: 37 - DNA, 25 bp, Artificial
  synthetic oligonucleotide
ctaagaatgtcaggaataggtcctg

SEQ ID NO: 38 - DNA, 24 bp, Artificial
  synthetic oligonucleotide
caccataggatagacatgcattcc

SEQ ID NO: 39 - DNA, 24 bp, Artificial
  synthetic oligonucleotide
gtacttagtggcaatgatggtgtc

SEQ ID NO: 40 - DNA, 23 bp, Artificial
  synthetic oligonucleotide
gcttgtaacagtgcccacatatc

SEQ ID NO: 41 - DNA, 23 bp, Artificial
  synthetic oligonucleotide
cgattatcagtgtcgagttgttc

SEQ ID NO: 42 - DNA, 22 bp, Artificial
  synthetic oligonucleotide
gtatgtcccccatgagatagtc

SEQ ID NO: 43 - RNA, 20 nt, maize fine streak virus
ugugugguuuuucccacugc

SEQ ID NO: 44 - RNA, 20 nt, maize fine streak virus
gcaguaaaaaaacggacaca

SEQ ID NO: 45 - protein, 17 aa, Artificial
  synthetic polypeptide
KRSSDGTGNVSKKKSRK

SEQ ID NO: 46 - protein, 17 aa, Artificial
  synthetic polypeptide
RKALTKASKALFKGKIK

SEQ ID NO: 47 - protein, 17 aa, Artificial
  synthetic polypeptide
KKEDKAEKATTEKRKRQ

SEQ ID NO: 48 - protein, 20 aa, Artificial
  synthetic polypeptide
MMARLVPCFTLALLLHLTEC

SEQ ID NO: 49 - protein, 23 aa, Artificial
  synthetic polypeptide
FIIKLVIGFTVGTIMLYISWIII

SEQ ID NO: 50 - DNA, 29 bp, Artificial
  synthetic oligonucleotide
cgcgaattcatgagtcaaagaactctgcg

SEQ ID NO: 51 - DNA, 31 bp, Artificial
  synthetic oligonucleotide
gacgcgtcgacttagtccccaatctccatct

SEQ ID NO: 52 - DNA, 35 bp, Artificial
  synthetic oligonucleotide
cgcggatcccatgaattacaaccgtttgaaattcg

SEQ ID NO: 53 - DNA, 35 bp, Artificial
  synthetic oligonucleotide
gccgggagctcttaaaattccatcgaggcaattcc

SEQ ID NO: 54 - DNA, 32 bp, Artificial
  synthetic oligonucleotide
acgccgtcgacatggatccagaaatctatgat

SEQ ID NO: 55 - DNA, 31 bp, Artificial
  synthetic oligonucleotide
ccgcctcgagtcaaataacatgaatcccctt

SEQ ID NO: 56 - DNA, 42 bp, Artificial
  synthetic oligonucleotide
ccggaattccctaagaagaagaggaaggttatgaacacgatt

SEQ ID NO: 57 - DNA, 32 bp, Artificial
  synthetic oligonucleotide
acgccgtcgaccgcgaacgcgaagtccgactc

SEQ ID NO: 58 - DNA, 48 bp, Artificial
  synthetic oligonucleotide
ccggaattctaatacgactcactataacacaggcaaaaaaatgacgca

SEQ ID NO: 59 - DNA, 42 bp, Artificial
  synthetic oligonucleotide
cccacagtccacccacctccatcatttgtacaattcatccat

SEQ ID NO: 60 - DNA, 42 bp, Artificial
  synthetic oligonucleotide
atggatgaattgtacaaatgatggaggtgggtggactgtggg

SEQ ID NO: 61 - DNA, 42 bp, Artificial
  synthetic oligonucleotide
taattcttcacctttagacatctatgtgtgggagttttggag

SEQ ID NO: 62 - DNA, 42 bp, Artificial
  synthetic oligonucleotide
ctccaaaactcccacacatagatgtctaaaggtgaagaatta

SEQ ID NO: 63 - DNA, 42 bp, Artificial
  synthetic oligonucleotide
gcgtcaccctttttggtgtgtgggtcggcatggcatctccac

SEQ ID NO: 64 - DNA, 42 bp, Artificial
  synthetic oligonucleotide
gtggagatgccatgccgacccacacaccaaaaagggtgacgc

SEQ ID NO: 65 - DNA, 40 bp, Artificial
  synthetic oligonucleotide
cgcctcgagcaaaaaacccctcaagacccgtttagaggcc

SEQ ID NO: 66 - DNA, 15 bp, Artificial
  synthetic oligonucleotide
tttattttgtagttg

SEQ ID NO: 67 - DNA, 15 bp, Artificial
  synthetic oligonucleotide
tttattttgtagtta

SEQ ID NO: 68 - DNA, 15 bp, Artificial
  synthetic oligonucleotide
ttgtttttgtagata

SEQ ID NO: 69 - DNA, 15 bp, Artificial
  synthetic oligonucleotide
caactacaaaataaa

SEQ ID NO: 70 - DNA, 15 bp, Artificial
  synthetic oligonucleotide
taactacaaaataaa

SEQ ID NO: 71 - DNA, 15 bp, Artificial
  synthetic oligonucleotide
tatctacaaaaacaa

(19) United States
(12) Patent Application Publication
(10) Pub. No.: US 2008/0020371 A1
(43) Pub. Date: Jan. 24, 2008

(54) Reverse Genetics System

(76) Inventors: Thomas Lotell German, Madison, WI (US); Paul Lawrence Flanary, Madison, WI (US); Saskia A. Hogenhout, Norwich (GB)

Correspondence Address: QUARLES & BRADY LLP, 411 E. WISCONSIN AVENUE, SUITE 2040, MILWAUKEE, WI, 53202-4497, US

(21) Appl. No.: 11/735,273

(22) Filed: Apr. 13, 2007

Related U.S. Application Data
(60) Provisional application No. 60/792,002, filed on Apr. 14, 2006.

(51) Int. Cl. C12Q 1/70; C07H 21/00; C07K 14/00; C07K 16/00; C12N 1/15; C12N 15/63; C12N 5/10

(52) U.S. Cl. 435/5; 435/254.2; 435/320.1; 435/348; 435/419; 435/69.1; 530/350; 530/387.9; 536/23.72

(57) ABSTRACT

The complete genomic sequence of maize fine streak virus (MFSV), a negative-strand RNA virus that infects plants and insects, is disclosed. The inventors have characterized the MFSV genome and identified the leader and trailer sequences, seven open reading frames as well as the functions of some of the encoded proteins, and the gene junction sequences and their functions. Using various functionally important components of the MFSV genome, the inventors have demonstrated that a reverse genetics system of using cloned cDNA to produce infectious viruses can be developed for plant negative-strand RNA viruses. Methods of using the reverse genetic system to produce wild-type or recombinant plant negative-strand RNA viruses, to express genes of interest, and to screen for agents that may affect the production and function of the viruses are disclosed. Further disclosed are various nucleic acid constructs, vectors, genetically engineered cells, and kits useful in the reverse genetics system.

### CROSS-REFERENCE TO RELATED APPLICATION

This application claims the benefit of U.S. Patent Application No. 60/792,002, filed on Apr. 14, 2006, which is herein incorporated by reference in its entirety.

### STATEMENT REGARDING FEDERALLY SPONSORED RESEARCH OR DEVELOPMENT

This invention was made with United States government support awarded by the following agency: USDA/CSREES 2002-35302-12653. The United States has certain rights in this invention.

### BACKGROUND OF THE INVENTION

The use of reverse genetics or the ability to produce infectious virus entirely from cloned cDNA has revolutionized the field of virology involving positive-strand RNA viruses. Knowledge concerning the important diseases caused by a large number of negative-strand RNA viruses has suffered for the lack of such systems. To date, only a few reverse-genetics systems exist for negative-strand RNA viruses of animals and there are no such systems for plant infecting viruses. What is needed in the art is a reverse genetics system based on plant-infecting, negative-strand RNA viruses. Such a system will provide new tools for, among other things, expressing exogenous genes and inducing gene silencing in plants, studying plant genetics and genetics of plant-infecting viruses, and producing wild-type or recombinant viruses and vaccines in plants.

Recent studies have shown that the plant system provides many practical, economic, and safety advantages compared with conventional systems for the production and convenient delivery of various pharmaceuticals such as protein- or peptide-based pharmaceuticals. Plants provide a natural, renewable resource for making proteins at a lower facility and production cost. In the case of subunit vaccines (Daniell, H. et al. (2001) Trends Plant Sci. 6:219-226; Ma, J. K.-C. et al. (2003) Nat. Rev. Genet. 4:794-805; Goldstein, D. A. & Thomas, J. A. (2004) Q. J. Med. 97:705-716; Gleba, Y. et al. (2004) Biotechnol. Genet. Eng. Rev. 21:325-367; and Koprowski, H. (2005) Vaccine 23:1757-1763), for example, vaccines produced by the plant system has been shown to be suitable for oral delivery to induce protective immune responses without injection-related hazards (Zhou, J. Y. et al. (2003) J. Virol. 77:9090-9093; Bae, J.-L. et al. (2003) Vaccine 21:4052-4058; Lamphear, B. J. et al. (2004) Vaccine, 22, 2420-2424; Ogra, P. L et al. (2001) Clin. Microbiol. Rev. 14:430-445; and Tuboly, T. et al. (2000) Vaccine 18:2023-2028). Reverse genetics systems based on plant-infecting, negative-strand RNA viruses will provide new tools for producing pharmaceuticals with plants.

### BRIEF SUMMARY OF THE INVENTION

The complete genomic sequence of maize fine streak virus (MFSV), a negative-strand RNA virus that infects plants and insects, is disclosed. The inventors have characterized the MFSV genome and identified the leader and trailer sequences, seven open reading frames as well as the functions of some of the encoded proteins, and the gene junction sequences and their functions. Using various functionally important components of the MFSV genome, the inventors have demonstrated that a reverse genetics system of using cloned cDNA to produce infectious viruses can be developed for plant negative-strand RNA viruses. Methods of using the reverse genetic system to produce wild-type or recombinant plant negative-strand RNA viruses, to express genes of interest, and to screen for agents that may affect the production and function of the viruses are disclosed. Further disclosed are various nucleic acid constructs, vectors, genetically engineered cells, and kits useful in connection with the reverse genetics system.

### BRIEF DESCRIPTION OF THE SEVERAL VIEWS OF THE DRAWINGS

FIG. 1 is a schematic diagram of the MFSV genome organization. The open reading frames (block arrows) are derived from the antigenomic sequence. The asterisks indicate gene junctions (FIG. 3). Bold lines indicate locations of probes A, B, C, D, E, F, and G used for Northern blot hybridization of mRNAs of ORF1, -2, -3, -4, -5, -6, and -7, respectively (FIG. 5).

FIG. 2 shows sequences of the 3′ and 5′ termini of plant rhabdovirus genomes. Sequences are shown in the genomic sense. Vertical lines indicate complementary nucleotides between leader and trailer sequences. Overhangs in leader sequences are underlined. The upper and lower MFSV sequences are provided in the sequence listing under SEQ ID NO:12 and SEQ ID NO:13, respectively. The upper and lower SYNV sequences are provided in the sequence listing under SEQ ID NO:14 and SEQ ID NO:15, respectively. The upper and lower RYSV sequences are provided in the sequence listing under SEQ ID NO:16 and SEQ ID NO:17, respectively. The upper and lower NCMV sequences are provided in the sequence listing under SEQ ID NO:18 and SEQ ID NO:19, respectively. The upper and lower LNYV sequences are provided in the sequence listing under SEQ ID NO:20 and SEQ ID NO:21, respectively.

FIG. 3 shows comparison of rhabdovirus gene junctions. (A) Gene junctions of the MFSV genome. The three motifs are indicated, corresponding to the 3′ ends of the mRNAs (column 1), intergenic sequences (column 2), and the 5′ ends of the mRNAs (column 3). (B) Consensus sequences of gene junctions of plant and animal rhabdoviruses. All sequences are presented in genomic sense in the 3′-to-5′ orientation, and nucleotide differences are underlined. Abbreviations: 3′le, 3′ leader sequence; 5′tr, 5′ trailer sequence; (N)ₙ, variable number of nucleotides. The sequence for 3′le/1, 1/2, 2/3, 4/5, 5/6, and 6/7 in panel A and for MFSV in panel B is provided in the sequence listing under SEQ ID NO:9. The sequence for 3/4 in panel A is provided in the sequence listing under SEQ ID NO:10. The sequence for 7/5′tr in panel A is provided in the sequence listing under SEQ ID NO:11. The sequence for SYNV in panel B is provided in the sequence listing under SEQ ID NO:22. The sequence for RYSV in panel B is provided in the sequence listing under SEQ ID NO:23. The sequence for NCMV in panel B is provided in the sequence listing under SEQ ID NO:24. The sequence for LNYV in panel B is provided in the sequence listing under SEQ ID NO:25. The sequence for VSV in panel B is provided in the sequence listing under SEQ ID NO:26. The sequence for RABV in panel B is provided in the sequence listing under SEQ ID NO:27.

FIG. 4 shows RLM-RACE (RNA ligase-mediated rapid amplification of cDNA ends) of the MFSV G and L mRNAs. The sequences of six clones, each derived from the G gene and L gene mRNAs, were determined. Nucleotides of mRNAs complementary to the transcription start sites in the conserved gene junction sequences of the viral genome (vg) are boxed. The vg RNA sequence in panel A is provided in the sequence listing under SEQ ID NO:28. The G mRNA 1-6 sequence in panel A is provided in the sequence listing under SEQ ID NO:29. The vg RNA sequence in panel B is provided in the sequence listing under SEQ ID NO:30. The mRNA 1-4 sequence in panel B is provided in the sequence listing under SEQ ID NO:31. The L mRNA 5 sequence in panel B is provided in the sequence listing under SEQ ID NO:32. The L mRNA 6 sequence in panel B is provided in the sequence listing under SEQ ID NO:33.

FIG. 5 shows the detection of MFSV gene transcripts in infected maize by Northern blot analysis. (A) Blots of total RNA isolated from healthy (data not shown) or MFSV-infected maize hybridized to probes A, B, C, D, E, F, and G, which are located within the MFSV N, P, 3, 4, M, G, and L genes, respectively. (B) Blot hybridized to probe D and subsequently to probe C. FIG. 1 shows the localizations of the probes. The asterisks indicate migration of the genomic RNA of MFSV, and the arrows indicate hybridization to mRNAs. Gel images of 25S rRNA were used as loading controls (below).

FIGS. 6-8 present a schematic illustration of an embodiment of the reverse genetics system of the present invention. The MFSV gene junction sequence shown in FIG. 8 is provided in the sequence listing under SEQ ID NO:66.

### DETAILED DESCRIPTION OF THE INVENTION

The present invention is based on the inventors' identification of functionally important genes and cis-acting sequences in maize fine streak virus (MFSV) and the demonstration that a reverse genetics system can be established with these genes and sequences. MFSV is a plant nucleorhabdovirus that is transmitted by the leafhopper Graminella nigrifrons (Hogenhout, S. A. et al. Trends Microbiol. 11:264-271, 2003; and Redinbaugh, M. G. et al. Phytopathology 92:1167-1174, 2002). Like those of other rhabdoviruses, the MFSV virion is a bacilliform particle measuring 231 by 71 nm with a lipid envelope, and its genome contains a nonsegmented, negative-sense, single-stranded RNA (Redinbaugh, M. G. et al. Phytopathology 92:1167-1174, 2002; and Walker, P. J. et al. Family Rhabdoviridae, p. 563-583. In M. H. V. van Regenmortel, et al. (ed.), Virus taxonomy: classification and nomenclature of viruses. Seventh report of the International Committee on Taxonomy of Viruses. Academic Press, San Diego, Calif. 2000). As shown in the examples below, the inventors have sequenced the entire MFSV genome and identified the leader and trailer sequences, seven open reading frames, and the corresponding gene junctions. The functions of 5 of the open reading frames have been identified. These 5 genes encode L, N, P, M, and G proteins, respectively. Using a yeast-GFP (green fluorescent protein) system, the inventors have demonstrated that a gene can be expressed using reverse genetics that involves flanking the gene at both ends with an MFSV gene junction sequence as well as an MFSV leader sequence and an MFSV trailer sequence and providing the L and N proteins. The expression can be enhanced if the P protein is also provided. The demonstration made with the yeast-GFP system shows that one can generate MFSV particles using a DNA sequence that is complementary to the MFSV genomic or antigenomic RNA. While the demonstration was made in yeast cells, it is expected that the same reverse genetics system would also work in MFSV's natural host cells, i.e., insect cells and plant cells (e.g., monocot plant cells).

While various methods, compositions, agents, and kits of the present invention are described in connection with MFSV below, it is noted that they can be practiced with any plant negative-strand RNA viruses (e.g., plant negative-strand RNA rhabdoviruses), including using the proteins, the leader and trailer sequences, and the gene junction sequences of these negative-strand RNA viruses. Examples of such plant negative-strand RNA viruses include but are not limited to Northern cereal mosaic virus (NCMV), Rice yellow stunt virus (RYSV), and Sonchus yellow net virus (SYNV). The genomic RNA sequences of these viruses are available in the art (the references are provided in Example 1 below). Another example is Lettuce necrotic yellows virus (LNYV).

The term negative-strand RNA virus refers to a negative-sense (antisense), single-stranded RNA virus wherein the negative-sense RNA is transcribed to create mRNA. The terms “viral genomic/genome RNA,” “negative-strand RNA,” “genomic-sense RNA,” and “negative-sense RNA” are used interchangeably in the application in connection with a negative-strand RNA virus to refer to the 3′ to 5′ genomic RNA of the negative-strand RNA virus such as MFSV that can be transcribed to mRNA molecules. The term “antigenomic RNA,” “viral complementary RNA (cRNA),” and “positive-strand RNA” are used interchangeably in the application in connection with a negative-strand RNA virus to refer to an RNA strand that is complementary to the 3′ to 5′ genomic RNA of the negative-strand RNA virus.

The terms MFSV leader sequence, MFSV trailer sequence, and MFSV gene junction sequences are used in the application to encompass both the genomic sequence and the antigenomic sequence. When used in the context of DNA constructs and vectors, these terms encompass the DNA sequences that correspond to the MFSV genomic and antigenomic sequences.

A DNA sequence corresponding to an RNA sequence or strand refers to a DNA sequence that is the same as the RNA sequence or strand except that any nucleotide “U” in the RNA sequence is replaced with a “T.”

The term “isolated nucleic acid” or “isolated polypeptide” used herein means a nucleic acid (e.g., DNA or RNA) or polypeptide isolated from its natural environment or prepared using synthetic methods such as those known to one of ordinary skill in the art. Complete purification is not required in either case. The nucleic acids or polypeptides of the invention can be isolated and purified from normally associated material in conventional ways such that in the purified preparation the nucleic acid or polypeptide is the predominant species in the preparation. At the very least, the degree of purification is such that the extraneous material in the preparation does not interfere with use of the nucleic acid or polypeptide of the invention in the manner disclosed herein. The nucleic acid or polypeptide is preferably at least about 85% pure, more preferably at least about 95% pure and most preferably at least about 99% pure.

Further, an isolated nucleic acid has a structure that is not identical to that of any naturally occurring nucleic acid. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of clones, e.g., as these occur in a DNA library such as a cDNA or genomic DNA library. An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine.

As used in this application, “percent identity” between amino acid or nucleotide sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993), or other methods noted in the application. The noted algorithm is incorporated into the NBLAST and XBLAST programs of Altschul et al. (J. Mol. Biol. 215, 403-410, 1990). BLAST nucleotide searches are performed with the NBLAST program, score=100, wordlength=12, to obtain nucleotide sequences homologous to a polynucleotide of the invention. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25, 3389-3402, 1997). When utilizing BLAST and Gapped BLAST programs, the default parameters of the respective programs (e.g., XBLAST and NBLAST) are used.

It is well known in the art that the amino acids within the same conservative group can typically substitute for one another without substantially affecting the function of a protein. For the purpose of the present invention, such conservative groups are set forth in Table 1 based on shared properties.

TABLE 1
Conservative substitution.

| Original Residue | Conservative Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr, Phe |
| Tyr (Y) | Trp, Phe, Thr, Ser |
| Val (V) | Ile, Leu, Met, Phe, Ala |

In one aspect, the present invention relates to an isolated polypeptide that contains (1) an MFSV protein identified by the inventors, (2) an MFSV protein with one or more conservative substitutions, or (3) an amino acid sequence having at least 90%, 95%, 97%, or 99% identity to an MFSV protein identified by the inventors. The MFSV proteins identified by the inventors include nucleocapsid protein or N protein (SEQ ID NO:2), phosphoprotein or P protein (polymerase transcription subunit, SEQ ID NO:3), SEQ ID NO:4, SEQ ID NO:5, matrix protein or M protein (SEQ ID NO:6), glycoprotein or G protein (SEQ ID NO:7), and polymerase or L protein (core polymerase, SEQ ID NO:8). A polypeptide closely related to an MFSV protein, i.e., an MFSV protein with one or more conservative substitutions or a polypeptide having at least 90%, 95%, 97%, or 99% identity to an MFSV protein at the amino acid level, and maintains the function of the MFSV protein, is referred to as a functional derivative of the MFSV protein.

In another aspect, the present invention relates to an isolated nucleic acid that contains a polynucleotide or a complement of the polynucleotide wherein the polynucleotide is an uninterrupted coding sequence for (1) an MFSV protein, (2) an MFSV protein with one or more conservative substitutions and preferably maintains the function of the MFSV protein, or (3) a polypeptide having at least 90%, 95%, 97%, or 99% identity to an MFSV protein at the amino acid level and preferably maintains the function of the MFSV protein. Full length MFSV genomic RNA and full length MFSV antigenomic RNA as well as the corresponding DNA molecules are specifically excluded from the nucleic acids of the present invention. In one embodiment, the MFSV protein-encoding polynucleotide is nucleotides 254-1642 of SEQ ID NO:1, 1946-2962 of SEQ ID NO:1, 3165-3446 of SEQ ID NO:1, 3532-4515 of SEQ ID NO: 1, 4744-5484 of SEQ ID NO:1, 5733-7523 of SEQ ID NO:1, or 7664-13498 of SEQ ID NO:1 (see GenBank Accession No. NC_{—}005974 or AY618417).

An isolated nucleic acid containing a polynucleotide (or its complement) having at least 90%, at least 95%, at least 97%, or at least 99% identity to any of the uninterrupted coding sequences described above is also within the scope of the present invention.

An isolated nucleic acid containing a polynucleotide (or its complement) that can hybridize to any of the uninterrupted coding sequences described above, under either stringent or moderately stringent hybridization conditions, is also within the scope of the present invention. Stringent hybridization conditions are defined as hybridizing at 68° C. in 5×SSC/5× Denhardt's solution/1.0% SDS, and washing in 0.2×SSC/0.1% SDS±100 μg/ml denatured salmon sperm DNA at room temperature, and moderately stringent hybridization conditions are defined as washing in the same buffer at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

In another aspect, the present invention relates to an expression vector containing any of the uninterrupted coding sequences described above (e.g., an MFSV protein-encoding polynucleotide) and a non-native promoter operably linked to the polynucleotide.

In another aspect, the present invention relates to a host cell containing any of the uninterrupted coding sequences described above (e.g., an MFSV protein-encoding polynucleotide) and a non-native promoter operably linked to the polynucleotide (e.g., a vector provided above). Examples of suitable host cells include plant cells (e.g., cells of a monocot plant), insect cells, and yeast cells.

In another aspect, the present invention relates to a reverse genetics system for generating wild-type MFSV or a recombinant MFSV in a cell or for expressing (either at the mRNA or protein level) a DNA sequence of interest (e.g., a gene) in a cell. The system employs DNA sequences that encode MFSV proteins, DNA sequences that correspond to MFSV gene junctions, and DNA sequences that correspond to MFSV leader and trailer sequences. Suitable cells include plant cells (e.g., cells of monocot plants), insect cells, and yeast cells. By recombinant MFSV, we mean an MFSV that has been manipulated in vitro, e.g., using recombinant DNA technology to introduce changes to the viral genome.

For generating wild-type MFSV, a DNA sequence complementary to the negative strand MFSV genomic RNA (vRNA) is provided in an expression vector wherein the DNA sequence is operably linked to a promoter and a terminator for the production of the negative-strand RNA. To ensure the correct ends of expressed viral genomic RNA, a ribozyme can be incorporated into the expression vector inside the terminator, the promoter, or both. A skilled artisan is familiar with the ribozymes such as a hammerhead ribozyme or an HDV ribozyme that can be used in connection with ensuring the correct 5′ or 3′ end of viral RNA. Said vector is then introduced into a permissive host cell that contains or is capable of producing a suitable DNA-dependent RNA polymerase for the synthesis of MFSV genomic RNA from the DNA sequence, the MFSV L and N proteins or functional derivatives thereof, and optionally, the MFSV P protein or a functional derivative thereof. The DNA-dependent RNA polymerase drives the synthesis of MFSV genomic RNA and the MFSV L and N proteins or their functional derivatives then form a complex with the MFSV genomic RNA to generate mRNAs and produce MFSV proteins. The MFSV genomic RNA is also used as a template to make full length MFSV antigenomic RNA (cRNA), which is in turn used to make more copies of the full length MFSV genomic RNA. Wild-type MFSV viral particles are next generated. It is noted that while only L and N proteins are needed for transcription (generating mRNA), all three of the L, N, and P proteins are needed for RNA replication (generating the full length viral genomic RNA and viral antigenomic RNA).

A skilled artisan is familiar with the DNA-dependent RNA polymerases that can be employed to synthesize the full length viral genomic RNA in the above reverse genetics system and can readily select suitable promoters and terminators to construct the vector. Examples of such polymerases include but are not limited to bacteriophage T7 polymerase, bacteriophage T3 polymerase, bacteriophage SP6 polymerase, and eukaryotic polymerase I. In one embodiment, the endogenous DNA-dependent RNA polymerases in plant cells, insect cells, and yeast cells are relied on to make MFSV genomic RNA. In this case, no exogenous DNA-dependent RNA polymerase needs to be introduced into the cells. The endogenous polymerases work with a variety of strong promoters such as Gal promoters (e.g., Gal10) and cauliflower mosaic virus 35S promoter. It is noted that while some DNA-dependent RNA polymerases such T7 polymerase can generate viral genomic RNA with a correct end, others such as the endogenous polymerases typically cannot. In the latter case, a ribozyme is provided downstream of the promoter and upstream of the leader sequence to produce the correct end.

The MFSV N and L proteins (or their functional derivatives), and if applicable, the MFSV P protein (or a functional derivative) and a suitable DNA-dependent RNA polymerase can be provided in the cell either through the use of expression vectors or by integrating the corresponding DNA sequences encoding the proteins into the cellular genome where the coding sequences are operably linked to a non-native promoter. When expression vectors are used, an expression vector can express one, two, three, or all four proteins or their functional equivalents. Such a vector typically contains a suitable promoter operably linked to a coding sequence. The promoter can be a constitutive, inducible, or tissue specific promoter. If the DNA-dependent RNA polymerase employed cannot readily enter the nucleus, a nucleus localization signal (NLS) such as SV40 NLS is provided.

In addition to the wild-type MFSV viral particles, recombinant MFSV particles can also be generated with the above system by introducing mutations (e.g., substitutions, deletions, and insertions) into the DNA sequence that is complementary to the negative strand MFSV genomic RNA. A recombinant MFSV may have an altered (e.g., attenuated or weakened) activity. Introducing mutations to the DNA sequence also provides a convenient way to study the activity and function of the MFSV genes as well as the gene junction sequences such as those important for generating mRNA, for recognition by viral polymerases, and for packaging signals necessary to generate a mature virion. One may also use the system to study the contribution of host genes to viral function such as virus replication. For example, on can use the system to make wild-type or recombinant MFSV in yeast cells with known gene deletions (such as a library of knock-outs that is commercially available) and look for cells that no longer support specific aspects of the viral life cycle such as replication, transcription, localization to specific subcellular compartments, or others.

The above system can also be used to express a DNA sequence of interest (e.g., to express an exogenous or heterologous gene in a plant cell, an insect cell, or a yeast cell). For example, one can make a full set of MFSV proteins in a plant cell, an insect cell, or a yeast cell using the vector containing a DNA sequence complementary to the full length MFSV genomic RNA described above. One can also make a subset of the MFSV proteins by deleting or interrupting the gene(s) encoding the other protein(s). A non-MFSV DNA sequence of interest (e.g., a non-MFSV gene) can be expressed by inserting the DNA sequence of interest into an MFSV gene (will generate a chimeric protein) or by replacing an MFSV gene with the DNA sequence of interest. In this case, MFSV proteins encoded by any remaining MFSV genes will also be made. One can also insert a non-MFSV DNA sequence of interest flanked by an MFSV gene junction sequence at both ends into the DNA sequence complementary to the full length MFSV genomic RNA between the leader and trailer sequences in a manner not to disrupt the expression of the MFSV proteins. This way, the non-MFSV DNA sequence will be expressed along with the MFSV proteins and viral particles can be generated to infect other cells which result in the expression of the non-MFSV DNA sequence of interest in other cells.

If one intends to express only non-MFSV DNA sequences of interest such as non-MFSV genes, all MFSV viral genes can be deleted and replaced by non-MFSV genes of interest while retaining the other elements of the vector. For example, such a vector would contain a promoter (and a ribozyme downstream of the promoter if applicable), an MFSV leader sequence, a gene of interest flanked by an MFSV gene junction sequence at both ends, an MFSV trailer sequence, a ribozyme if applicable downstream of the trailer sequence, and a terminator. More than one non-MFSV genes can be expressed from one vector by including more than one gene of interest flanked by MFSV gene junction sequences in the vector. For example, a total of 2 to 10, 2 to 7, or 2 to 5 genes can be expressed from one vector. A gene that is located closer to the leader sequence should express at a higher level than a gene that is located father away from the leader sequence. Regardless of the number and type of genes one wishes to express, each gene should be flanked on both sides by an MFSV gene junction sequence corresponding to one of SEQ ID NOs:9-11 or a complement of one of SEQ ID NOs:9-11 (TTTATTTTGTAGTTG, SEQ ID NO:66; TTTATTTTGTAGTTA, SEQ ID NO:67; TTGTTTTTGTAGATA, SEQ ID NO:68; CAACTACAAAATAAA, SEQ ID NO:69; TAACTACAAAATAAA, SEQ ID NO:70; and TATCTACAAAAACAA, SEQ ID NO:71). These gene junctions sequences provide the signals for starting and stopping transcription. An MFSV leader sequence (e.g., nucleotides 1-184 of SEQ ID NO:1 or its complement) and an MFSV trailer sequence (e.g., nucleotides 13638-13782 of SEQ ID NO:1 or its complement) are retained so that the full length RNA can be copied back and forth between the negative strand and the positive strand. Similar to the vector for making the wild-type MFSV, a negative-strand RNA spanning from the leader sequence to the trailer sequence with correct ends is made by the DNA-dependent RNA polymerase from the vector for expression non-MFSV genes. MFSV N and L proteins provided in the host cell form a complex with the negative strand to make mRNA of the gene(s) of interest and the host cell translation machinery then synthesizes the corresponding protein(s). The full length negative-strand RNA is also copied to make a full length positive-strand RNA and vise versa by the MFSV N, L, and P proteins provided in the host cell, which can enhance the expression efficiency by providing more copies of the negative-strand RNA for making mRNA.

It is noted that a gene or DNA sequence of interest to be expressed using the reverse genetic system described above is provided in antisense so that the corresponding mRNA cannot be made unless the antisense sequence is first copied by the MFSV L protein or a functional derivative thereof. cDNAs from a plant cDNA library can be expressed in plant cells using the system disclosed here to study gene function. The system can also be used to make a short RNA for the silencing or decreasing of the expression of a gene in a cell. Further, the system can be used to make vaccines (e.g., subunit vaccines), which may be edible vaccines.

While the reverse genetic system is described above in connection with the use of an expression vector to initially make or launch a negative-strand RNA, it is understood that an expression vector containing a DNA sequence that is complementary to the corresponding positive-strand RNA and thus would initially launch the positive strand can also be used to generate wild-type or recombinant MFSV particles or to express a DNA sequence of interest in plant cells, insect cells, or yeast cells. This is because the MFSV N, L, and P proteins provided in the cells will copy the positive strand to make the negative strand. Other aspects of the system are the same as the system for initially launching a negative-strand RNA and they are within the scope of the present invention.

Another application of the reverse genetics system disclosed here is to identify candidate agents that can potentially modulate MFSV activity (e.g., by modulating production and/or infection efficiency). In one embodiment, cells that can generate wild-type MFSV or a recombinant MFSV are exposed to a test agent and the generation of wild-type MFSV or the recombinant MFSV is determined and compared to that of control cells not exposed to the test agent. A higher or lower level of viral particle production in the test agent-treated group than the control group indicates that the test agent can modulate the activity of MFSV. In one form, the system is used to identify candidate agents that can potentially inhibit the activity of MFSV.

In another embodiment, cells that can express a DNA sequence of interest (e.g., a gene encoding a protein such as GFP) are exposed to a test agent and the expression level of the DNA sequence of interest is determined and compared to that of control cells not exposed to the test agent. A higher or lower expression level in the test agent-treated group than the control group indicates that the test agent can modulate the activity of MFSV. In one form, the system is used to identify candidate agents that can potentially inhibit the activity of MFSV.

In another aspect, the present invention relates to various agents and compositions described above in connection with the reverse genetics system. In this regard, one embodiment of the present invention relates to a cell having at least one coding sequence for one of the following proteins stably integrated into its genome and the coding sequence is under the control of a non-native promoter such that the proteins can be expressed in the cell. These proteins include the MFSV N protein (SEQ ID NO:2) or a functional derivative thereof, the MFSV L protein (SEQ ID NO:8) or a functional derivative thereof, the MFSV P protein (SEQ ID NO:3) or a functional derivative thereof, and a DNA-dependent RNA polymerase.

In another embodiment, the present invention relates to kit that contains a nucleic acid having a nucleotide sequence that encodes the MFSV N protein (SEQ ID NO:2) or a functional derivative thereof and a nucleic acid having a nucleotide sequence that encodes the MFSV L protein (SEQ ID NO:8) or a functional derivative thereof. The kit can optionally contain a nucleic acid having a nucleotide sequence that encodes the MFSV P protein (SEQ ID NO:3) or a functional derivative thereof. The kit can also optionally contain a nucleic acid having a nucleotide sequence that encodes a DNA-dependent RNA polymerase. All the nucleic acids can optionally contain a non-native promoter operably linked to the coding sequences. Further, the kit can optionally contain a vector for generating wild-type MFSV or a recombinant MFSV in a cell or for expressing a DNA sequence of interest in a cell described above. Such a vector may contain one or more multiple cloning sites so that one or more DNA sequences or genes of interest can be inserted into the vector for expression in plant cells, insect cells, or yeast cells.

By way of example, but not limitation, examples of the present invention are described below.

### EXAMPLE 1

### Complete Genome Sequence and in Planta Subcellular Localization of Maize Fine Streak Virus Proteins

This example shows that the genome of the nucleorhabdovirus maize fine streak virus (MFSV) contains 13,782 nucleotides of nonsegmented, negative-sense, single-stranded RNA. The antigenomic strand contains seven open reading frames (ORFs), and transcripts of all ORFs were detected in infected plants. ORF1, ORF2, ORF5, ORF6, and ORF7 encode the nucleocapsid protein (N), phosphoprotein (P), matrix protein (M), glycoprotein (G), and polymerase (L), respectively. The ORF1(N), ORF4, and ORF5 (M) proteins localized to nuclei, consistent with the presence of nuclear localization signals (NLSs) in these proteins. The ORF2 (P) protein spread throughout the cell when expressed alone but was relocalized to a subnuclear locus when coexpressed with the MFSV N protein. Unexpectedly, coexpression of the MFSV N and P proteins, but not the orthologous proteins of SYNV, resulted in accumulations of both proteins in the nucleolus. The N and P protein relocalization was specific to cognate proteins of each virus.

Members of the family Rhabdoviridae have a broad host range, including humans, livestock, plants, and insects. Six genera of rhabdoviruses have been described (40). The four genera of animal-infecting rhabdoviruses (Vesiculovirus, Ephemerovirus, Lyssavirus, and Novirhabdovirus) include the livestock pathogens Vesicular stomatitis Indiana virus (VSIV), Vesicular stomatitis New Jersey virus (VSNJV), and Bovine ephemeral fever virus; the human pathogen Rabies virus (RABV); and the fish pathogen Infectious hematopoietic necrosis virus. The two genera of plant rhabdoviruses are Cytorhabdovirus (type species, Lettuce necrotic yellows virus [LNYV]) and Nucleorhabdovirus (type species, Potato yellow dwarf virus). The viruses that cause rabies and fish diseases appear to be confined to vertebrate hosts, whereas vesiculo-, ephemero-, cyto-, and nucleorhabdoviruses are transmitted to their vertebrate or plant hosts by insects (20, 35). Plant rhabdoviruses are particularly interesting because they are able to replicate and systemically spread in very divergent hosts: plants and insects.

Generally, a rhabdovirus virion is composed of a lipid envelope derived from host membranes and a ribonucleocapsid core consisting of a nonsegmented, negative-sense, single-stranded RNA bound to complexes of nucleocapsid protein (N), phosphoprotein (P), and polymerase (L) (35). The glycoprotein (G) protrudes from the exterior of the lipid envelope, and the matrix protein (M) connects the envelope to the ribonucleocapsid core (35). VSIV and VSNJV have the simplest genomes, encoding just the five structural proteins of the virion in the gene order 3′-N-P-M-G-L-5′, whereas the genomes of other rhabdoviruses harbor additional genes (18, 26, 30, 39, 42).

More than 70 plant rhabdoviruses have been described and the genomes of several plant rhabdoviruses have been sequenced to completion, including Northern cereal mosaic virus (NCMV), Rice yellow stunt virus (RYSV), and Sonchus yellow net virus (SYNV) (4, 5, 6, 9, 11, 17, 19, 21, 28, 29, 36, 39, 41, 43, 44, 45). The nucleorhabdovirus SYNV is presently the most extensively characterized plant-infecting rhabdovirus (22). In planta subcellular localization studies of fluorescent-protein fusions provided an indication of the function and protein-protein interactions of viral proteins of SYNV (13). The SYNV N and M proteins both target the plant cell nucleus when expressed individually, whereas the SYNV P and sc4 proteins do not (13). However, the SYNV P protein targets subnuclear locales when coexpressed with the SYNV N protein, suggesting that the N and P proteins interact with each other in SYNV-infected plants (12, 13). The SYNV sc4 gene, located between the P and M genes, encodes a membrane-associated protein that may be involved in viral cell-to-cell movement in plants (31, 36).

Maize fine streak virus (MFSV) was first reported in maize fields in southwestern Georgia in 1999 and was described as a new plant nucleorhabdovirus (34). The symptoms caused by MFSV include dwarfing and fine chlorotic streaks along intermediate and small veins (34). MFSV is transmitted by the leafhopper Graminella nigrifrons and is not transmissible by rub inoculation of maize leaves but can be mechanically transmitted by vascular puncture inoculation (VPI) (20, 34). Like those of other rhabdoviruses, the MFSV virion is a bacilliform particle measuring 231 by 71 nm with a lipid envelope, and its genome consists of a nonsegmented, negative-sense, single-stranded RNA (34, 40). Purified preparations of MFSV contain three abundant proteins corresponding to the major rhabdovirus structural proteins: the G protein (82 kDa), N protein (50 kDa), and M protein (32 kDa) (34).

To define MFSV genes and to begin to characterize their functions, we determined the complete genomic sequence of the virus and investigated virus gene expression in infected maize. We showed for the first time the localization of coexpressed rhabdoviral N and P proteins in the nucleolus of plant cells and that the N and P proteins of MFSV and SYNV specifically interact with each other and not with the orthologous proteins of another rhabdovirus.

Materials and Methods

Virus maintenance and purification: MFSV from Georgia was maintained in maize seedlings by serial inoculations with viruliferous insect vectors (G. nigrifrons) or by VPI (27, 34). The virus was purified from maize leaf laminar tissue collected 26 to 40 days after VPI or vector inoculation, as previously described (34).

MFSV genomic RNA extraction and library construction: For initial cDNA library construction, virus genomic RNA was extracted using a previously described protocol (34). For all other applications (reverse transcription [RT]-PCR, 3′ rapid amplification of cDNA ends (RACE), 5′ RACE, and RNA ligase-mediated (RLM) RACE), genomic RNA was extracted from virus pellet suspensions using a ToTALLY RNA isolation kit (Ambion, Austin, Tex.) following the manufacturer's instructions.

The cDNA synthesis of the MFSV genomic RNA using random hexamers was carried out with the Superscript Choice system (Invitrogen Corp., Carlsbad, Calif.) according to the manufacturer's instructions. The cDNAs were ligated into the EcoRI-digested, phosphatase-treated pGEM4Z vector (Promega Corp., Madison, Wis.) or the pZeroAmp vector (T. Meulia, unpublished results) and transformed into Escherichia coli TOP 10 cells (Invitrogen Corp.). Twelve clones (G2A, G2C, G3A, G3B, G4C, G5C, G6A, G9B, Z6B, Z11B, Z12B, and Z15B) carrying inserts larger than 1 kb that hybridized with viral RNA were selected for sequence analysis.

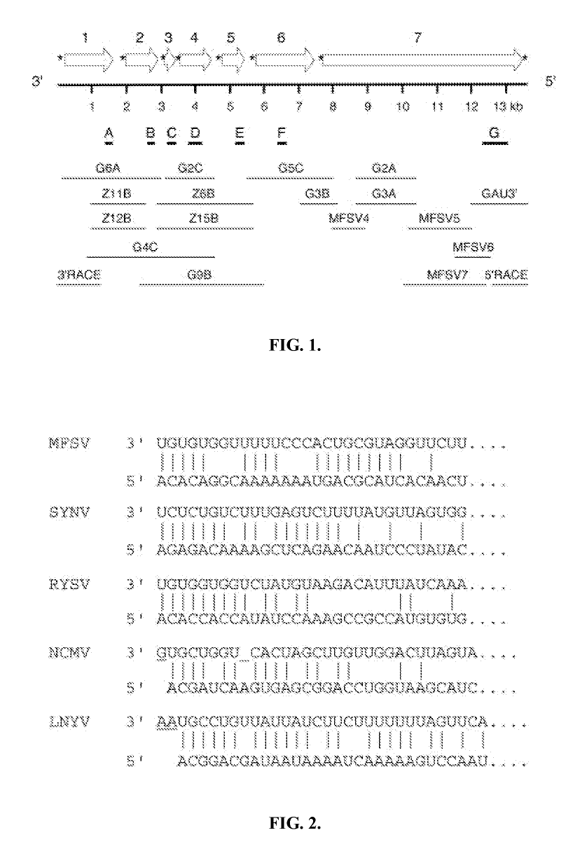

Regions of the MFSV genomic RNA not represented by clones were amplified using RT-PCR with primers flanking the missing sequence. RT-PCR was carried out using a Platinum RT-PCR kit (Invitrogen Corp.). RT-PCR products for the 3′ end of the MFSV genome (GAU3′) and MFSV4 (FIG. 1) were ligated into the pCR-Blunt II-TOPO vector (Invitrogen Corp.) for sequencing. Three other RT-PCR products, MFSV5, MFSV6, and MFSV7 (FIG. 1) were sequenced directly, without prior cloning.

MFSV genome sequencing and sequence analysis: Sequencing was carried out in 550- to 700-bp segments by primer walking using a 3700 DNA Sequence Analyzer and BigDye Terminator Cycle Sequencing chemistry (Applied Biosystems, Inc., Foster City, Calif.) according to the supplier's instructions. Base calling was done with MacPHRED-MacPHRAP (CodonCode Corp., Dedham, Mass.), and sequences were assembled by Sequencher (Gene Codes Corp., Ann Arbor, Mich.).

ORFs were identified with MacVector version 6.5 (Accelrys, San Diego, Calif.). Putative MFSV protein sequences were compared to the National Center for Biotechnology Information (NCBI) GenBank database by BLASTP search to identify sequence similarity to other known proteins. Protein sequences were searched for domains and motifs, including transmembrane domains (TMHMM version 2.0 [http://www.cbs.dtu.dk/services/TMHMM/]) (37), N-terminal signal peptides (SignalP version 3.0 [http://www.cbs.dtu.dk/services/SignalP/]) (3), nuclear localization signals (NLSs) (PROSITE [http://us.expasy.org/prosite/] (8) and PredictNLS [http://cubic.bioc.columbia.edu/predictNLS/] (7)), and glycosylation sites (PROSITE).

Sequencing of the 3′ and 5′ ends of the MFSV genome: The terminal sequences of MFSV were identified by RACE. The viral 5′ trailer region was determined by both 5′ RACE (Invitrogen Corp.) and RLM-RACE (Ambion) following the manufacturers′ instructions. For 5′ RACE, the cDNA of the MFSV genomic RNA was synthesized using a gene-specific primer, 5′ RACE GSP1 (5′-AAATCTCTGTTGAGCC-3′, SEQ ID NO:34), and tailed with dCTP using terminal deoxynucleotidyl transferase. The first amplification was carried out with the abridged anchor primer and a 5′ RACE GSP2 primer (5′-GGTCCATTGCAGAGAGATCAAC-3′, SEQ ID NO:35). Nested amplification used the abridged universal amplification primer (AUAP) and a 5′ RACE GSP3 primer (5′-CTATCCTATCAGATCCCATAATGC-3′, SEQ ID NO:36). For RLM-RACE, a 45-bp 5′ RACE adapter was added to the MFSV genomic RNA, and then the cDNA was synthesized using random decamers. The first amplification was carried out with the 5′ RACE outer primer and the 5′ RACE GSP2 primer. Nested amplification used the 5′ RACE inner primer and the 5′ RACE GSP3 primer.

The viral 3′ leader region was determined by 3′ RACE (Invitrogen Corp.). The MFSV genomic RNA was tailed with ATP using the Poly(A) Tailing kit (Ambion), and cDNA was synthesized using the oligo(dT)-containing adapter primer. The first amplification was carried out with the AUAP and a 3′ RACE GSP1 primer (5′-CTAAGAATGTCAGGAATAGGTCCTG-3′, SEQ ID NO:37), and nested amplification was done with the AUAP and a 3′ RACE GSP2 primer (5′-CACCATAGGATAGACATGCATTCC-3′, SEQ ID NO:38). The 5′ RACE and 3′ RACE products were ligated into the pGEM-T Easy vector (Promega Corp.) for sequencing.

Rhabdovirus nucleotide sequences used for interspecies comparison were obtained from the genome sequences in the NCBI GenBank database for the following viruses: SYNV (NC_{—}001615), RYSV (NC_{—}003746), NCMV (NC_{—}002251), LNYV (L24365 and L24364) (42), VSIV (NC_{—}001560), and RABV (NC_{—}001542).

Characterization of transcription start sites of the MFSV G and L genes: The transcription start sites of the MFSV G and L genes were identified by RLM-RACE. Total RNA from infected maize leaves was extracted with a ToTALLY RNA isolation kit. Subsequently, mRNA was isolated from the total RNA preparation using a Dynabead mRNA DIRECT kit (Dynal ASA, Oslo, Norway) following the manufacturer's instructions. The cDNA was synthesized from the adapter-ligated mRNA with random decamers. The first amplification was carried out with either the 5′ RACE outer primer and a G outer primer (5′-GTACTTAGTGGCAATGATGGTGTC-3′, SEQ ID NO:39) or the 5′ RACE outer primer and an L outer primer (5′-GCTTGTAACAGTGCCCACATATC-3′, SEQ ID NO:40). Nested amplification was carried out with either the 5′ RACE inner primer and a G inner primer (5′-CGATTATCAGTGTCGAGTTGTTC-3′, SEQ ID NO:41) or the 5′ RACE inner primer and an L inner primer (5′-GTATGTCCCCCATGAGATAGTC-3′, SEQ ID NO:42). The RLM-RACE products were ligated into the pGEM-T Easy vector for sequencing.

Northern blot hybridization analysis: Total RNA (10 μg) from infected and healthy maize was denatured using glyoxal sample loading dye (Ambion) and separated on a 1.2% BPTE {100 mM PIPES [piperazine-N,N′-bis(2-ethanesulfonic acid)], 300 mM bis-Tris, 10 mM EDTA, pH 6.5} agarose gel, transferred to a positively charged BrightStar-Plus nylon membrane (Ambion) with 20×SSC (3 M NaCl, 0.3 M sodium citrate, pH 7.0), and cross-linked to the membrane by exposure to UV light (UV Transilluminator; Fisher Scientific, Pittsburgh, Pa.) for 2 min. Probes were prepared by PCR amplification of DNA fragments corresponding to each gene from the cDNA clones and subsequent incorporation of [³²P]dCTP (Amersham Biosciences Corp., Piscataway, N.J.) (10). Hybridization of probes to Northern blots was carried out as described in reference 32. Northern blots were washed three times for 10 min each in 2×SSC and 0.1% sodium dodecyl sulfate and four times for 10 min each in 0.1×SSC and 0.1% sodium dodecyl sulfate at 65° C. and then exposed to Storage Phosphor Screen (Molecular Dynamics, Sunnyvale, Calif.) for 24 h. Images were captured using ImageQuant software (Molecular Dynamics), converted to TIFF for export, and processed in Photoshop version 7.0 (Adobe, San Jose, Calif.).

Construction of pGD derivatives for in planta subcellular localization: Construction of the binary pGDG and pGDR vectors, the pGDG construct for in planta synthesis of the Arabidopsis thaliana Fib1 (AtFib1)-green fluorescent protein (GFP), GFP-SYNV N, and the pGDR construct for in planta synthesis of DsRed-SYNV P were described in reference 13. The pGDB and pGDY vectors were constructed by modification of pGD (13). The cyan fluorescent protein (CFP) and yellow fluorescent protein (YFP) genes were amplified from pECFP-C1 and pEYFP-C1 (BD Biosciences, Palo Alto, Calif.), respectively. The PCR products were subcloned into the pGD vectors in a manner that reconstituted the multiple cloning site of pGD, since BglII, HindIII, and XhoI restriction sites were incorporated into the 3′ ends of the PCR products. The CFP and YFP genes in the new vectors were verified by DNA sequencing and expression in plant cells. The complete predicted MFSV ORFs (i.e., from the putative start codon to the first stop codon) were expressed as fusions to the C termini of autofluorescent proteins. The MFSV ORFs were amplified from corresponding cDNA clones by PCR, and primers with overhanging restriction sites were introduced to facilitate directional cloning into the binary vector pGDB, pGDG, pGDR, or pGDY as described in reference 13. PCR was performed using the high-fidelity DynazymeEXT polymerase (Finnzymes, Espoo, Finland). PCR products were cloned directly into the pCRII vector (Invitrogen Corp.) using topoisomerase-mediated cloning. The DNA sequences of the full-length clones for the MFSV N, P, 3, 4, and M genes in pGD derivatives were verified prior to transformation into Agrobacterium tumefaciens strain C58C1.

Agroinfiltration procedures: A. tumefaciens was infiltrated into leaves of Nicotiana benthamiana as described in reference 13. Briefly, suspensions of transformed C58C1 agrobacteria were adjusted to an optical density at 600 nm of 0.6 in agroinfiltration buffer (10 mM MgCl₂, 10 mM MES [morpholineethanesulfonic acid], pH 5.6), and acetosyringone was added to a final concentration of 150 μM (13). An agrobacterial suspension was infiltrated into the mesophyll of leaves using a 1-ml disposable syringe. Following infiltration, the leaves were examined by epifluorescence microscopy between 40 and 90 h postinfiltration.

DAPI staining of plant nuclei: DAPI (4′-6-diamidino-2-phenylindole dihydrochloride; 15 μg/ml) in agroinfiltration buffer was infiltrated into leaves as described by Goodin et al. (13). Following infiltration, the plants were incubated in the dark for 1 to 2 h before examination of leaf sections by epifluorescence microscopy.

Epifluorescence microscopy: Epifluorescence micrographs were acquired using an Axiocam MR monochromatic digital camera mounted on a motorized Axioplan2 microscope (Carl Zeiss Microimaging Inc., Thornwood, N.Y.). Camera and microscope settings were controlled by Axiovision software version 4.1. False colors for differentiating DAPI, CFP, GFP, and DsRed2 fluorescences were assigned using color settings in the Axiovision software. Filter sets that permitted viewing of the relevant fluorescences were purchased from Chroma Technology Corp. (Rockingham, Vt.) and included the following filter sets. (i) Filter set 31001 for viewing GFP; this set consisted of a D470/40× excitation (Ex) filter, a 505 DCLP dichroic, and a D540/40× emission (Em) filter. (ii) Filter set 31000 was used for viewing DAPI-stained nuclei. This set consisted of a D360/40× Ex filter, a 400 DCLP dichroic, and a D460/50 M Em filter. (iii) Filter set 310044 V2, used for capturing CFP fluorescence, consisted of a D436/20× Ex filter, a 455DCLP dichroic, and a D480/40 M Ex filter. (iv) YFP fluorescence was viewed using a 41028 filter set that consisted of an HQ500/20× Ex filter, a Q5151LP dichroic, and a HQ535/30 M Em filter. (v) For viewing fluorescence from DsRed2, a 41035 filter set, consisting of a HQ546/12× Ex filter, a Q560LP dichroic, and a HQ650/75 M Em filter, was used. The lenses used in this study included the Plan Neofluar 10×/NA 0.3; the Plan Neofluar 25×/NA 0.8 multiple immersion lens, used primarily in the water immersion setting; and a Plan Apochromat 100×/NA 1.4 oil immersion lens. Sections of plant tissue were mounted in water on standard glass slides and covered with no. 1 glass coverslips (Coming Inc., Coming, N.Y.). Leaves were mounted so that the abaxial surface was viewed. Micrographs were exported from the Axiovision software as TIFF files. All subsequent cropping and image manipulations were carried out in Photoshop version 7.0 (Adobe Systems Inc., San Jose, Calif.) and Canvas version 8.0 (Deneba Software, Miami, Fla.).

Confocal laser scanning microscopy: Confocal laser scanning micrographs were acquired on a TCS SP2-AOBS microscope (Leica Microsystems, Bannockburn, Ill.). GFP variants were excited simultaneously using the 488-nm laser line. Fluorescence emissions from CFP, GFP, and YFP could be distinguished using the spectral imaging capability provided by the prism spectrophotometer of the microscope. The ability to unambiguously differentiate these three fluors when coexpressed in leaf epidermal cells allowed assignment of the subnuclear locales in which the N and P proteins accumulate.

Results

Nucleotide sequence of MFSV: The MFSV genome sequence was obtained by assembling sequences derived from the overlapping sequences of 12 randomly primed cDNA clones, five RT-PCR products, and two RACE products (FIG. 1). The MFSV genome consisted of 13,782 nucleotides (nt) and seven ORFs on the antigenomic strand.

The sequence of the 3′ end of the MFSV genome was determined by 3′ RACE, and that of the 5′ end was determined by 5′ RACE and RLM-RACE. The 3′ RACE PCR products were cloned, and inserts of all six clones were identical, indicating that the 3′ end of the MFSV genome was 3′-UGUGUGGUUUUUCCCACUGC . . . -5′ (SEQ ID NO:43). The sequences of inserts of four 5′ RACE and RLM-RACE clones were identical, indicating that the 5′ end of the MFSV genome was 3′- . . . GCAGUAAAAAAACGGACACA-5′ (SEQ ID NO:44). Identical 5′- and 3′-end sequences using either poly(A) or poly(C) adapters were obtained at the University of Wisconsin (P. Flanary and T. L. German). These results led to the conclusion that the MFSV genomic RNA had a 184-nt 3′ leader region preceding the leader-N intergenic sequence and a 145-nt 5′ trailer region following the L-trailer intergenic sequence.

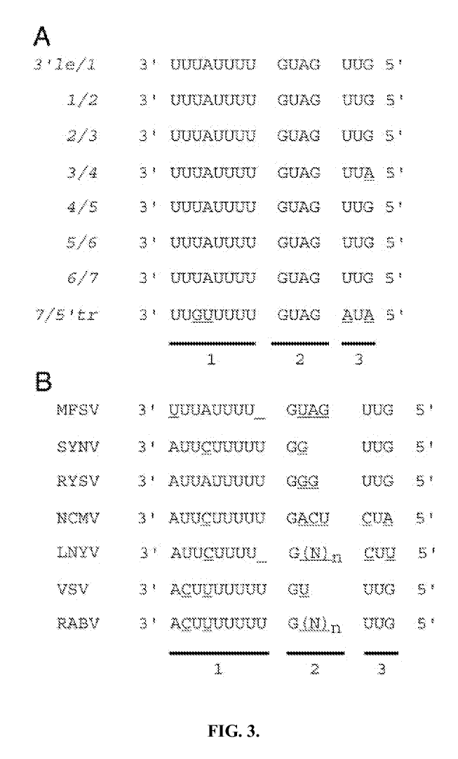

Comparison of the 3′ and 5′ ends of the MFSV genomic RNA revealed that 19 of 30 nucleotides were complementary and might give rise to a putative panhandle structure (FIG. 2). Similar structures were reported for SYNV, LNYV, and NCMV (6, 39, 42). In addition, the first 30 nt of the 3′ leader sequence had a high U residue content (47%), similar to those described for SYNV (53%), RYSV (37%), NCMV (37%), and LNYV (60%). Although the plant rhabdoviruses share complementarity of their 3′ and 5′ ends and have similar nucleotide biases, these sequences did not have significant sequence identity among rhabdoviruses (FIG. 2).

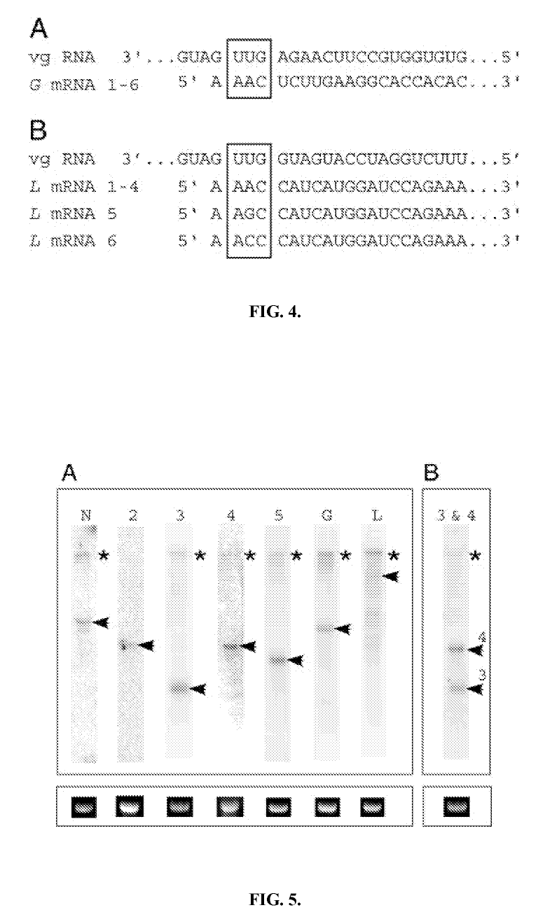

Gene junctions of the MFSV genome: The MFSV ORFs were separated by gene junctions with the consensus sequence 3′-UUUAUUUUGUAGUUG-5′ (SEQ ID NO:9) (FIG. 3A). This sequence was broadly conserved among plant and animal rhabdoviruses (FIG. 3B) and was divided into three distinct motifs: the sequences corresponding to the 3′ ends of mRNAs, the intergenic sequences, and the sequences corresponding to the 5′ ends of mRNAs (35) (FIG. 3). The corresponding sequences of MFSV mRNA 3′ ends were A/U rich and 8 nucleotides in length, and only the ORF7-5′ trailer junction had 2 nucleotide differences from the consensus sequence. The intergenic sequence GAUG was conserved in all gene junctions of MFSV; however, this sequence was not conserved among rhabdoviruses (FIG. 3B). The transcription start site consisted of 3 nucleotides, and only the ORF4 mRNA start site had a single-nucleotide change relative to the consensus sequence, UUG. The MFSV transcription start site was identical to those of other rhabdoviruses (SYNV, RYSV, VSIV, and RABV), except for the two cytorhabdoviruses NCMV and LNYV (FIG. 3B).

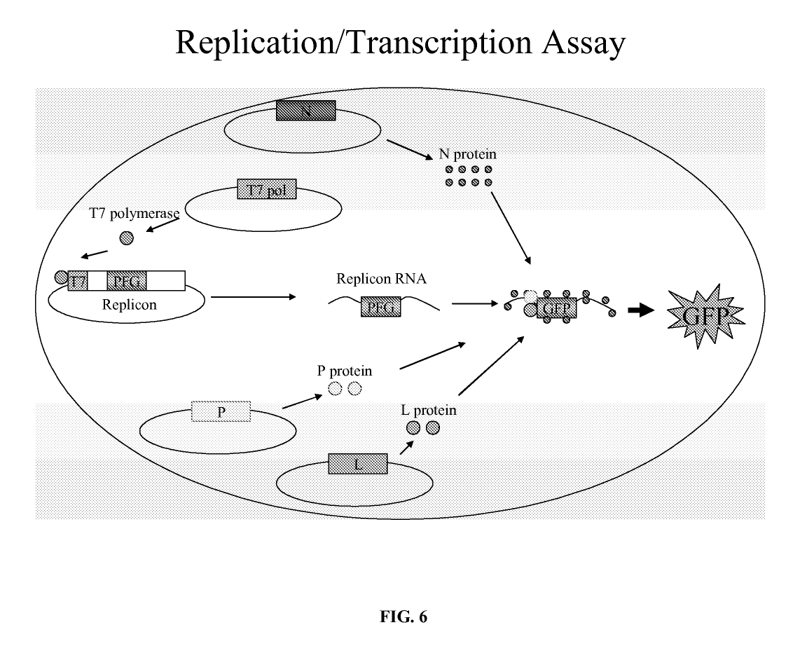

Transcription start sites of the MFSV G and L genes: RLM-RACE was used to confirm the sequences of the putative transcription start sites for the MFSV G and L genes (FIG. 4). The sequences of six RLM-RACE products for the G gene were identical (FIG. 4A). The sequences of four RLM-RACE products for the L gene were identical, while the sequences of two others differed from these at position 3 (FIG. 4B). These data are consistent with the putative UUG transcription start sites identified in FIG. 3A. However, the 5′ ends of transcripts contained a nucleotide A that does not correspond to the viral genomic sequence (FIG. 4).

Analysis of the MFSV ORF sequences: The predicted proteins encoded by the seven MFSV ORFs were examined for sequence similarity to other proteins in the NCBI GenBank nonredundant database using BLASTP. The deduced protein sequence of ORF1 had significant similarity to the N proteins of RYSV (expected [E] value=8e⁻²¹), SYNV (E value=1e⁻¹⁹), and NCMV (E value=5e⁻⁶). The ORF6 protein sequence had significant similarity to those of the G proteins of RYSV (E value=5e⁻²²) and SYNV (E value=1e⁻¹⁶), and the ORF7 protein sequence had significant similarity to those of the L proteins of SYNV (E value=0.0), RYSV (E value=0.0), and other nonsegmented, negative-sense RNA viruses. Thus, based on sequence similarity, we concluded that MFSV ORF1, ORF6, and ORF7 encode the N, G, and L proteins, respectively. The ORF2, ORF3, ORF4, and ORF5 protein sequences did not show any significant similarities to other rhabdovirus proteins or other sequences in GenBank.

The protein sequences of MFSV and other nucleorhabdoviruses were searched for domains and motifs, including NLSs, glycosylation sites, N-terminal signal peptides, and transmembrane domains. Putative NLSs were found at amino acid positions 436 to 452 (KRSSDGTGNVSKKKSRK, SEQ ID NO:45) of the N protein, at positions 17 to 33 (RKALTKASKALFKGKIK, SEQ ID NO:46) of the ORF4 protein, and at positions 195 to 211 (KKEDKAEKATTEKRKRQ, SEQ ID NO:47) of the ORF5 protein, whereas no NLSs were identified in the MFSV ORF2, ORF3, G, and L proteins (Table 2). The SYNV N and M and RYSV N proteins also had putative NLSs in the carboxyl regions, but no putative NLS was identified in the RYSV M protein (33).

TABLE 2
Features of encoded proteins of MFSV genome

| | Calculated | TM (position | NLS (position | |
|---|---|---|---|---|
| ORF | mass | in | in | |
| no. | (kDa) | protein)^{a} | protein)^{a} | Function |
| 1 | 51.6 | ND | +(C-term) | Nucleocapsid |
| | | | | protein |
| | | | | (N) |
| 2 | 38.4 | ND | ND | Phosphoprotein (P) |
| 3 | 10.7 | ND | ND | Unknown |
| 4 | 37.2 | ND | +(N-term) | Unknown |
| 5 | 28.5 | ND | +(C-term) | Matrix protein (M) |
| 6 | 67.0 | +(C-term) | ND | Glycoprotein |
| | | | | (G) |
| 7 | 223.5 | ND | ND | Polymerase |
| | | | | (L) |

^{a}TM, transmembrane domain; ND, not detected; N-term, N-terminal half of protein; C-term, C-terminal half of protein; +, present.

Similar to other rhabdovirus G proteins, the putative MFSV G protein apparently has abundant glycosylation signals. Eight potential glycosylation signals (N-[P]-S/T-[P]-) were located at amino acid positions 64 to 67, 131 to 134, 132 to 135, 139 to 142, 204 to 207, 325 to 328, 438 to 441, and 494 to 497. The MFSV G protein also had an N-terminal signal peptide sequence (MMARLVPCFTLALLLHLTEC, SEQ ID NO:48) and a putative cleavage site (C/A) between amino acid positions 20 and 21. Furthermore, a transmembrane domain (FIIKLVIGFTVGTIMLYISWIII, SEQ ID NO:49) was identified at amino acid positions 529 to 551. Other predicted proteins of MFSV did not have abundant glycosylation sites, signal peptide sequences, or transmembrane domains.

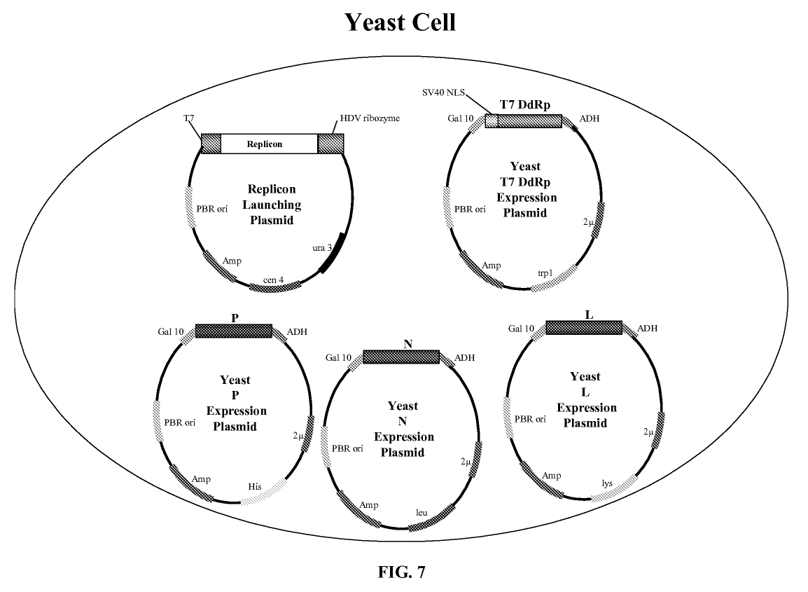

Detection of MFSV ORF transcripts in plants: To test whether all the identified ORFs in the MFSV genome are transcribed, Northern blots of total RNAs from healthy and MFSV-infected maize were hybridized with probes corresponding to each ORF (FIG. 1). Transcripts of expected sizes that corresponded to each of the seven ORFs were detected in MFSV-infected maize (FIG. 5), whereas no hybridization occurred with RNA from healthy maize (data not shown). Several RNAs hybridized to the ORF7 probe, with the size of the largest matching the expected size of the ORF7 transcript (FIG. 5A, lane L). The predicted length of gene 3 is 357 nt, and a transcript of about 400 bp hybridized to the ORF3 probe (FIG. 5A, lane 3). The predicted length of gene 4 is 1,185 nt, and a transcript of about 1,200 bp hybridized to the ORF4 probe (FIG. 5A, lane 4). To exclude the possibility of RNA degradation, a blot that was hybridized with the ORF4 probe was subsequently probed with the ORF3 probe. As expected, the resulting blot showed two distinct hybridized bands (FIG. 5B). These results demonstrated that seven distinct transcripts were present in MFSV-infected maize and that these transcripts corresponded to the seven putative genes identified in the MFSV genome.

Subcellular localization of the MFSV proteins: NLSs were identified in three of the seven MFSV ORFs, and nuclear localization was demonstrated for the SYNV N and M proteins (13). To determine the subcellular localization of the MFSV proteins, we used in planta localization and colocalization of fluorescent-protein fusions. Full-length ORF sequences were introduced into binary pGD derivatives for A. tumefaciens-mediated agroinfiltration of N. benthamiana leaves (13) and subsequent in planta production of MFSV proteins fused at the N terminus to CFP, YFP, and GFP. Epifluorescence micrographs and confocal micrographs were obtained. The DNA selective dye DAPI was used to determine the positions of nuclei in plant cells.

Agroinfiltration of fluorescent-protein fusions of MFSV N, ORF2, ORF3, ORF4, and ORF5 proteins showed that CFP-MFSV N, GFP-MFSV 4, and GFP-MFSV 5 accumulated in the nucleus, whereas YFP-MFSV 2 spread throughout the cell and YFP-MFSV 3 accumulated in punctate loci in the cytoplasm. These results were consistent with the prediction of NLSs in the MFSV N, ORF4, and ORF5 proteins.

When leaves were coinfiltrated with mixtures of C58C1 agrobacteria harboring CFP-MFSV N and YFP-MFSV 2 fusions, both fusions colocalized to the subnucleus. However, unlike the SYNV N and P proteins, the MFSV N and ORF2 proteins localized to the nucleolus. To confirm the nucleolar localization, we coexpressed CFP-MFSV N and YFP-MFSV 2 with the AtFib1-GFP that localizes to the nucleolus in A. thaliana and N. benthamiana epidermal cells (2, 13). This result confirmed that CFP-MFSV N and YFP-MFSV 2 targeted the same subnuclear locale as AtFib1-GFP, which is in the nucleolus.

Finally, we determined whether coinfiltration of fluorescent-protein fusions of the MFSV N protein and the SYNV P protein allowed redirection of cytosolic SYNV P protein to the subnuclear locale, similar to coexpression of the SYNV N and P proteins. Coinfiltrated CFP-MFSV N and DsRed-SYNV P did not interact to target a subnuclear locale but looked very similar to DsRed-SYNV P infiltrated alone. Similar results were obtained when GFP-SYNV N was coinfiltrated with YFP-MFSV 2. These data suggest that the interaction of N and P proteins leading to subnuclear targeting is virus specific.

### REFERENCES

1. Ahmed, M., M. O. McKenzie, S. Puckett, M. Hojnacki, L. Poliquin, and D. S. Lyles. 2003. Ability of the matrix protein of vesicular stomatitis virus to suppress beta interferon gene expression is genetically correlated with the inhibition of host RNA and protein synthesis. J. Virol. 77:4646-4657.

2. Barneche, F., F. Steinmetz, and M. Echeverria. 2000. Fibrillarin genes encode both a conserved nucleolar protein and a novel small nucleolar RNA involved in ribosomal RNA methylation in Arabidopsis thaliana. J. Biol. Chem. 275:27212-27220.

3. Bendtsen, J. D., H. Nielsen, G. von Heijne, and S. Brunak. 2004. Improved prediction of signal peptides: SignalP 3.0. J. Mol. Biol. 340:783-795.

4. Chen, X. Y., Z. L. Luo, and R. X. Fang. 1998. Structure analysis of the rice yellow stunt rhabdovirus gene 3. Chinese Sci. Bull. 43:745-748.

5. Choi, T. J., S. Kuwata, E. V. Koonin, L. A. Heaton, and A. O. Jackson. 1992. Structure of the L (polymerase) protein gene of sonchus yellow net virus. Virology 189:31-39.

6. Choi, T. J., J. D. Wagner, and A. O. Jackson. 1994. Sequence analysis of the trailer region of sonchus yellow net virus genomic RNA. Virology 202:33-40.

7. Cokol, M., R. Nair, and B. Rost. 2000. Finding nuclear localization signals. EMBO Rep. 1:411-415.

8. Falquet, L., M. Pagni, P. Bucher, N. Hulo, C. J. Sigrist, K. Hofmann, and A. Bairoch. 2002. The PROSITE database, its status in 2002. Nucleic Acids Res. 30:235-238.

9. Fang, R. X., Q. Wang, B. Y. Xu, Z. Pang, H. T. Zhu, K. Q. Mang, D. M. Gao, W. S. Qin, and N. H. Chua. 1994. Structure of the nucleocapsid protein gene of rice yellow stunt rhabdovirus. Virology 204:367-375.

10. Feinberg, A. P., and B. Volgelstein. 1983. A technique for radiolabeling DNA restriction endonuclease fragments to high specific activity. Anal. Biochem. 132:6-13.

11. Goldberg, K. B., B. Modrell, B. I. Hillman, L. A. Heaton, T. J. Choi, and A. O. Jackson. 1991. Structure of the glycoprotein gene of sonchus yellow net virus, a plant rhabdovirus. Virology 185:32-38.

12. Goodin, M. M., J. Austin, R. Tobias, M. Fujita, C. Morales, and A. O. Jackson. 2001. Interactions and nuclear import of the N and P proteins of sonchus yellow net virus, a plant nucleorhabdovirus. J. Virol. 75:9393-9406.

13. Goodin, M. M., R. G. Dietzgen, D. Schichnes, S. Ruzin, and A. O. Jackson. 2002. pGD vectors: versatile tools for the expression of green and red fluorescent protein fusions in agroinfiltrated plant leaves. Plant J. 31:375-383.

14. Gupta, A. K., D. Shaji, and A. K. Banerjee. 2003. Identification of a novel tripartite complex involved in replication of vesicular stomatitis virus genome RNA. J. Virol. 77:732-738.

15. Hanson, M. R., and R. H. Kohler. 2001. GFP imaging: methodology and application to investigate cellular compartmentation in plants. J. Exp. Bot. 52:529-539.

16. Harty, R. N., J. Paragas, M. Sudol, and P. Palese. 1999. A proline-rich motif within the matrix protein of vesicular stomatitis virus and rabies virus interacts with WW domains of cellular proteins: implications for viral budding. J. Virol. 73:2921-2929.

17. Heaton, L. A., D. Zuidema, and A. O. Jackson. 1987. Structure of the M2 protein gene of sonchus yellow net virus. Virology 161:234-241.

18. Heaton, L. A., B. I. Hillman, B. G. Hunter, D. Zuidema, and A. O. Jackson. 1989. Physical map of the genome of sonchus yellow net virus, a plant rhabdovirus with six genes and conserved gene junction sequences. Proc. Natl. Acad. Sci. USA 86:8665-8668.

19. Hillman, B. I., L. A. Heaton, B. G. Hunter, B. Modrell, and A. O. Jackson. 1990. Structure of the gene encoding the Ml protein of sonchus yellow net virus. Virology 179:201-207.

20. Hogenhout, S. A., M. G. Redinbaugh, and E.-D. Ammar. 2003. Plant and animal rhabdovirus host range: a bug's view. Trends Microbiol. 11:264-271.

21. Huang, Y., H. Zhao, Z. Luo, X. Chen, and R. X. Fang. 2003. Novel structure of the genome of Rice yellow stunt virus: identification of the gene 6-encoded virion protein. J. Gen. Virol. 84:2259-2264.

22. Jackson, A. O., M. Goodin, I. Moreno, J. Jackson, and D. M. Lawrence. 1999. Plant rhabdoviruses, p. 1531-1541. In A. Granoff and R. G. Webster (ed.), Encyclopedia of virology. Academic Press, San Diego, Calif.

23. Jayakar, H. R., and M. A. Whitt. 2002. Identification of two additional translation products from the matrix (M) gene that contribute to vesicular stomatitis virus cytopathology. J. Virol. 76:8011-8018.

24. Kopecky, S. A., and D. S. Lyles. 2003. Contrasting effects of matrix protein on apoptosis in HeLa and BHK cells infected with vesicular stomatitis virus are due to inhibition of host gene expression. J. Virol. 77:4658-4669.

25. Krug, R. M. 1981. Priming of influenza viral RNA transcription by capped heterologous RNAs. Curr. Top. Microbiol. Immunol. 93:125-149.

26. Kurath, G., K. H. Higman, and H. V. Bjorklund. 1997. Distribution and variation of NV genes in fish rhabdoviruses. J. Gen. Virol. 78:113-117.

27. Louie, R. 1995. Vascular puncture of maize kernels for the mechanical transmission of maize white line mosaic virus and other viruses of maize. Phytopathology 85:139-143.

28. Luo, Z., X. Chen, D. Gao, and R. Fang. 1998. The gene 4 of rice yellow stunt rhabdovirus encodes the matrix protein. Virus Genes 16:277-280.

29. Luo, Z. L., and R. X. Fang. 1998. Structure analysis of the rice yellow stunt rhabdovirus glycoprotein gene and its mRNA. Arch. Virol. 143:2453-2459.

30. McWilliam, S. M., K. Kongsuwan, J. A. Cowley, K. A. Byrne, and P. J. Walker. 1997. Genome organization and transcription strategy in the complex G_{NS}-L intergenic region of bovine ephemeral fever rhabdovirus. J. Gen. Virol. 78:1309-1317.

31. Melcher, U. 2000. The ‘30K’ superfamily of viral movement proteins. J. Gen. Virol. 81:257-266.

32. Redinbaugh, M. G., G. J. Wadsworth, and J. G. Scandalios. 1988. Characterization of catalase transcripts and their differential expression in maize. Biochem. Biophys. Acta 951:104-116.

33. Redinbaugh, M. G., and S. A. Hogenhout. 2005. Plant rhabdoviruses, p. 143-163. In Z. Fu and H. Koprowski (ed.), Current topics in microbiology and immunology, vol. 292. The world of rhabdoviruses. Springer-Verlag, New York, N.Y.

34. Redinbaugh, M. G., D. L. Seifers, T. Meulia, J. J. Abt, R. J. Anderson, W. E. Styer, J. Ackerman, R. Salomon, W. Houghton, R. Creamer, D. T. Gordon, and S. A. Hogenhout. 2002. Maize fine streak virus, a new leafhopper-transmitted rhabdovirus. Phytopathology 92:1167-1174.

35. Rose, J. K., and M. A. Whitt. 2001. Rhabdoviridae: the virus and their replication, p. 1221-1244. In D. M. Knipe and P. M. Howely (ed.), Fields virology. Lippincott Williams & Wilkins, Philadelphia, Pa.

36. Scholthof, K. B., B. I. Hillman, B. Modrell, L. A. Heaton, and A. O. Jackson. 1994. Characterization and detection of sc4: a sixth gene encoded by sonchus yellow net virus. Virology 204:279-288.

37. Sonnhammer, E. L. L., G. von Heijne, and A. Krogh. 1998. A hidden Markov model for predicting transmembrane helices in protein sequences, p. 175-182. In J. Glasgow, T. Littlejohn, F. Major, R. Lathrop, D. Sankoff, and C. Sensen (ed.), Proceedings of the Sixth International Conference on Intelligent Systems for Molecular Biology. AAAI Press, Menlo Park, Calif.

38. Stewart, C. N. 2001. The utility of green fluorescent protein in transgenic plants. Plant Cell Rep. 20:376-382.

39. Tanno, F., A. Nakatsu, S. Toriyama, and M. Kojima. 2000. Complete nucleotide sequence of northern cereal mosaic virus and its genome organization. Arch. Virol. 145:1373-1384.

40. Walker, P. J., A. Benmansour, R. Dietzgen, R. X. Fang, A. O. Jackson, G. Kurath, J. C. Leong, S. Nadin-Davies, R. B. Tesh, and N. Tordo. 2000. Family Rhabdoviridae, p. 563-583. In M. H. V. van Regenmortel, C. M. Fauquet, D. H. L. Bishop, E. B. Carstens, M. K. Estes, S. M. Lemon, J. Maniloff, M. A. Mayo, D. J. McGeoch, C. R. Pringle, and R. B. Wickner (ed.), Virus taxonomy: classification and nomenclature of viruses. Seventh report of the International Committee on Taxonomy of Viruses. Academic Press, San Diego, Calif.

41. Wang, Q., X. Y. Chen, Z. L. Luo, and R. X. Fang. 1999. Sequence analysis of leader and trailer regions of rice yellow stunt rhabdovirus and characterization of their in vivo transcripts. Sci. China Ser. C 42:50-56.

42. Wetzel, T., R. G. Dietzgen, and J. L. Dale. 1994. Genomic organization of lettuce necrotic yellows rhabdovirus. Virology 200:401-412.

43. Zhu, H. T., X. Y. Chen, Z. L. Luo, R. X. Fang, and D. M. Gao. 1997. Nucleotide sequence of the rice yellow stunt rhabdovirus gene 2. Chinese J. Virol. 13:369-375.

44. Zuidema, D., L. A. Heaton, R. Hanau, and A. O. Jackson. 1986. Detection and sequence of plus-strand leader RNA of sonchus yellow net virus, a plant rhabdovirus. Proc. Natl. Acad. Sci. USA 83:5019-5023.

45. Zuidema, D., L. A. Heaton, and A. O. Jackson. 1987. Structure of the nucleocapsid protein gene of Sonchus yellow net virus. Virology 159:373-380.

### EXAMPLE 2

### Negative-Strand RNA Virus Vector for Improved Expression in Plants and Insects

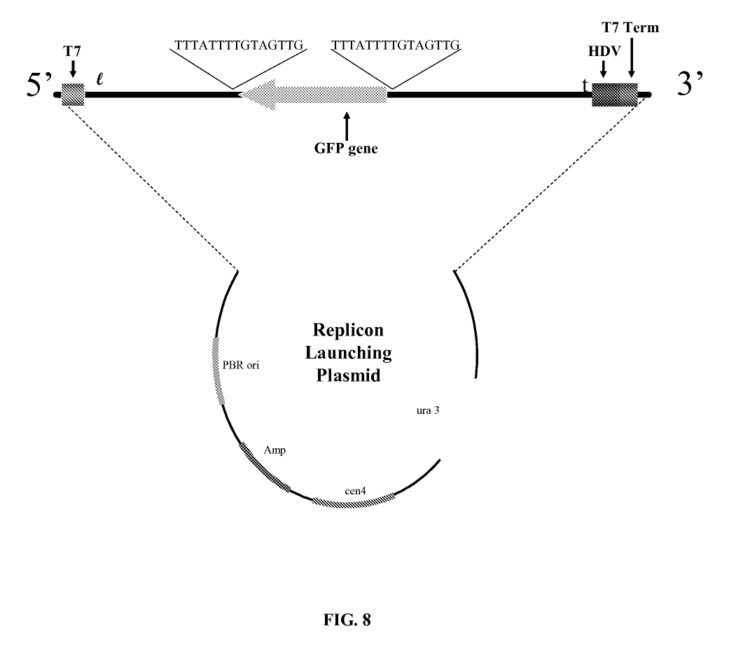

FIGS. 6-8 present a schematic illustration of an embodiment of the reverse genetics system of the present invention. As shown in FIG. 6, expression plasmids/vectors capable of expressing MFSV N, L, and P proteins and T7 DNA-dependent RNA polymerase (T7 pol or T7 DdRp) are provided in a yeast cell (FIG. 7 shows the structure of these plasmids in detail). FIG. 6 further shows that a replicon launching plasmid for the expression of GFP protein is also provided in the yeast cell. “GFP” is written backwards to indicate that it is in the negative sense and cannot produce protein (or report) unless it is copied by the MFSV L protein (FIG. 6). FIGS. 7 and 8 show the detailed structure of the replicon launching plasmid and replicon. The replicon represents a minimal subset of the viral genome with the cis acting nucleotide signals required for replication and transcription. The replicon is operably linked to a T7 promoter (T7) and a T7 terminator (T7 Term) as well as a HDV ribozyme (HDV) inside the terminator. The replicon itself contains an MFSV leader sequence (1), a DNA sequence of interest to be expressed (the gfp gene) flanked at both ends by an MFSV gene junction sequence (TTTATTTTGTAGTTG, SEQ ID NO:66), an MFSV trailer sequence(t).

Upon expression of T7 DNA-dependent RNA polymerase as well as the MFSV N, L, and P proteins in the yeast cell, a replicon RNA is made by the T7 DNA-dependent RNA polymerase and the N, L, and P proteins then bind to the replicon RNA to form a biologically active transcription/replication complex that leads to the expression of the GFP protein (FIG. 6).

The experimental details below show that the inventors have implemented the system described above to successfully express the gfp gene embedded in the MFSV replicon in yeast cells. The MFSV N and P proteins were found to have accumulated at similar sites in yeast and plant cells (data not shown).

Standard methods for the routine cultivation, maintenance, manipulation of DNA and RNA, and transformation of yeast and bacteria were used throughout (Ausubel, F. M., R. Brent, R. E. Kingston, D. D. Moore, J. G. Seidman, J. A. Smith, and K. Struhl (ed.). 1987. Current protocols in molecular biology. Wiley Interscience, New York, N.Y.). The yeast Saccharomyces cerivisiae strain used in this study was a trp1 deletion strain of BY4741, 4007202 (MATα his3Δ leu2Δ met15Δ ura3Δ trp1::G418^{R}) (ATCC).

Vector Construction: The bi-directional yeast GAL1/10 promoter was isolated from pBS-GAL (pBS was obtained from Stratagene and Gal template was from pRS316-GAL obtained form Dr. Henrik Dohlman, Dohlman, H. G. et al., Mol Cell Biol., 1995, 15:3635-43) as a BamHI-EcoRI fragment and subcloned into the matching sites of pRS414 and pRS416 (all pRS vectors were obtained from ATCC) to yield pRS414GAL and pRS416GAL. A BamHI-SalI fragment was isolated from pBS-GAL and subcloned into the matching sites of pRS411 and pRS415 to yield pRS411GAL and pRS415GAL. The native T7 and T3 promoter sites of the pRSGAL constructs and pRS413 were deleted by site directed mutagenesis. The MFSV N, P and L open reading frames as well as T7 DNA-dependent RNA polymerase open reading frame were amplified using the primers listed in Table 3, and subcloned into the matching sites of the pRS vectors to yield pRS411Gal-N (N protein expression plasmid), pRS414Gal-T7DdRp (T7 DNA-dependent RNA polymerase expression plasmid), pRS415Gal-L (L protein expression plasmid), and pRS416Gal-P (P protein expression plasmid). The MFSV N, P and L PCR templates were generated by Reverse Transcription-PCR from purified viral RNA.

TABLE 3

Oligonucleotide primers used to construct plasmids.
Boldface sequences: restriction enzyme recognition sites;
italicized sequence: a SV40 nuclear localization signal.

| | |
|---|---|
| Primer | Primer Sequence |

| | |
|---|---|
| MFSV-P Forward | 5′-CGCGAATTCATGAGTCAAAGAACTCTGCG-3′ |
| (SEQ ID NO:50) | EcoRI |
| MFSV-P Reverse | 5′-GACGCGTCGACTTAGTCCCCAATCTCCATCT-3′ |
| (SEQ ID NO:51) | SalI |
| MFSV-N Forward | 5′-CGCGGATCCCATGAATTACAACCGTTTGAAATTCG-3′ |
| (SEQ ID NO:52) | BamHI |
| MFSV-N Reverse | 5′-GCCGGGAGCTCTTAAAATTCCATCGAGGCAATTCC-3′ |
| (SEQ ID NO:53) | SacI |
| MFSV-L Forward | 5′-ACGCCGTCGACATGGATCCAGAAATCTATGAT-3′ |
| (SEQ ID NO:54) | SalI |
| MFSV-L Reverse | 5′-CCGCCTCGAGTCAAATAACATGAATCCCCTT-3′ |
| (SEQ ID NO:55) | XhoI |
| T7DdRp Forward | 5′-CCGGAATTCCCTAAGAAGAAGAGGAAGGTTATGAACACGATT-3′ |
| (SEQ ID NO:56) | EcoRI SV40NLS |
| T7DdRp Reverse | 5′-ACGCCGTCGACCGCGAACGCGAAGTCCGACTC-3′ |
| (SEQ ID NO:57) | SalI |

The pRS413-REP (replicon launching plasmid) was generated by Overlap Extension PCR (OE-PCR) using the primers listed in Table 4 (Sambrook J, Fritsch E F, and Maniatis T: Molecular cloning a laboratory manual. Plainview, N.Y.: Cold Spring Harbor Press 1989). OE-PCR generates PCR fragments that have overlapping sequences and can subsequently be joined together by a second round of PCR in which the first pair of products function as both template and primers. The resultant product was cloned into pRS413 as a EcoRI-XhoI fragment to generate pRS413REP. The replicon consists of complementary DNA containing green fluorescent protein (GFP) as a reporter in the antisense orientation flanked by the MFSV untranslated 5′ trailer sequence and 3′ leader sequence. At the 5′ end of the replicon we incorporated a modified T7 promoter (modification consists of removing the final three G's of the published sequence), and at the 3′ end the self-cleaving HDV ribozyme (Kuo M Y et al. J Virol., 1988, 62:4439-4444; and Sharmeen L et al. J Virol., 1988, 62:2674-2679), such that generated transcript would contain the exact initiation and termination sequences of the native MFSV RNA. When observed for GFP production, it was observed that unintended transcription of GFP occurred from an undetermined cryptic promoter, so an NsiI-XhoI fragment was deleted from pRS413REP, and replaced with the yeast CYC1 transcriptional terminator (Saccharomyces Genome Database) to generate pRS413REPCYC (replicon launching plasmid).

TABLE 4

Oligonucleotide primers used for construction of the replicon launching
plasmid by Overlap Extension PCR. Regions of interest are indicated below
the primer sequences.

| | |
|---|---|
| Primer | Primer Sequence |

| | |
|---|---|
| T7PTF | 5′-CCGGAATTCTAATACGACTCACTATAACACAGGCAAAAAAATGACGCA-3′ |
| (SEQ ID NO:58) | EcoRI Modified T7 Promoter MFSV Transcriptional Terminator |
| GLTR | 5′-CCCACAGTCCACCCACCTCCATCATTTGTACAATTCATCCAT-3′ |
| (SEQ ID NO:59) | MFSV Transcriptional Terminator GFP Sequence |
| GLTF | 5′-ATGGATGAATTGTACAAATGATGGAGGTGGGTGGACTGTGGG-3′ |
| (SEQ ID NO:60) | GFP Sequence MFSV Transcriptional Terminator |
| NLGR | 5′-TAATTCTTCACCTTTAGACATCTATGTGTGGGAGTTTTGGAG-3′ |
| (SEQ ID NO:61) | GFP Sequence MFSV Transcriptional Initiator |
| NLGF | 5′-CTCCAAAACTCCCACACATAGATGTCTAAAGGTGAAGAATTA-3′ |
| (SEQ ID NO:62) | MFSV Transcriptional Initiator GFP Sequence |
| LHDVF | 5′-GCGTCACCCTTTTTGGTGTGTGGGTCGGCATGGCATCTCCAC-3′ |
| (SEQ ID NO:63) | MFSV Transcriptional Initiator HDV Ribozyme Sequence |
| HDVLR | 5′-GTGGAGATGCCATGCCGACCCACACACCAAAAAGGGTGACGC-3′ |
| (SEQ ID NO:64) | HDV Ribozyme Sequence MFSV Transcriptional Initiator |
| T7TERMPREV | 5′-CGCCTCGAGCAAAAAACCCCTCAAGACCCGTTTAGAGGCC-3′ |
| (SEQ ID NO:65) | XhoI T7 Transcriptional Terminator |

Localization Studies: Since the wild-type virus replicates in the nucleus of plant cells, we conducted comparative localization studies in yeast. We used the GFP plasmids pUG35 (C-tagged products) and pUG36 (N-tagged products). Both N-tagged and C-tagged products were constructed for MFSV N, P and M. The empty plasmid controls were located ubiquitously throughout the yeast. MFSV N-GFP (whether N-tagged or C-tagged) was localized in the yeast nucleus. MFSV P-GFP was localized to a sub-nuclear region of the yeast for only N-tagged products. The MFSV M-GFP products are localized ubiquitously in yeast, whether expressed alone, or co-expressed with N and/or P. However only N, P, and L are essential for the reverse genetics system, and failure of M to localize to the nucleus should not present a problem.

Replicon Expression: The replicon contains the viral antisense transcriptional (re-)initiation and termination sequences flanking GFP, driven by a T7 promoter modified to allow the transcription product to have the exact viral nucleotide at the 5′ end. An HDV ribozyme and a T7 transcriptional terminator were incorporated into the construct to ensure that an exact 3′ end of the viral genome is obtained as well, because it is well known that viral promoters must contain the precise viral sequences to function. Overlap Extension PCR (OE-PCR) was used to create this chimeric sequence construct. The replicon was sequenced and found to have mutations in six regions. The mutations were corrected by site-directed mutagenesis in a sequential fashion. The replicon was shown to be expressed under the control of T7 DNA-dependent RNA polymerase by Northern Blot analysis.

Reverse Genetics: To test the viability of the yeast based MFSV reverse genetics system, the following controls and treatments have been transformed into yeast and assayed for GFP expression:

Treatment 1: N protein expression plasmid alone;

Treatment 2: P protein expression plasmid alone;

Treatment 3: L protein expression plasmid alone;

Treatment 4: T7 DNA-dependent RNA polymerase expression plasmid+replicon launching plasmid;

Treatment 5: T7 DNA-dependent RNA polymerase expression plasmid+replicon launching plasmid+N protein expression plasmid;

Treatment 6: T7 DNA-dependent RNA polymerase expression plasmid+replicon launching plasmid+N protein expression plasmid+L protein expression plasmid; and

Treatment 7: T7 DNA-dependent RNA polymerase expression plasmid+replicon launching plasmid+N protein expression plasmid+L protein expression plasmid+P protein expression plasmid.

All of the single transformants (treatments 1, 2 and 3), as well as transformants of treatment 4 and treatment 5, showed no discernible growth defects. However, tranformants of treatment 6 grew very poorly in galactose media, and growth in galactose media was lethal for transformants of treatment 7. The lethality of the induced L when co-transformed with the other plasmids is in agreement with Sonchus yellow net virus, a close relative of MFSV. To overcome the lethality, we grew the cultures in dextrose media to mid-log, harvested yeast cells by centrifugation and subsequently resuspended the cells in fresh galactose media followed by two hours incubation.

Treatments 1-6 are important controls for nonspecific reporter activity, and the only combination that should report is treatment 7. However, it has been shown in animal rhabdovirus systems that P may not be essential. Therefore, treatment 6 could report as well. We observed that the replicon could be translated in the presence of the entire set of plasmids (=treatment 7). However two of the controls (treatments 4 and 5) had GFP production. We discovered that this was caused by a cryptic promoter on the 3′ side of our replicon construct that could be read by an endogenous yeast polymerase. To correct this problem we made a deletion of a large non-essential portion of the replicon launching plasmid to remove the problematic sequences (XhoI-NsiI fragment). The modified replicon launching plasmid did not report in yeast in the absence of appropriate viral proteins and the experiments were repeated with the modified replicon launching plasmid and the following control and treatment groups:

Treatment a): five empty plasmids (with no coding sequences for N protein, L protein, P protein, T7 DNA-dependent RNA polymerase, and GFP, respectively);

Treatment b): replicon launching plasmid alone;

Treatment c): replicon launching plasmid+T7 DNA-dependent RNA polymerase expression plasmid;

Treatment d): N protein expression plasmid+L protein expression plasmid+P protein expression plasmid;

Treatment e): replicon launching plasmid+T7 DNA-dependent RNA polymerase expression plasmid+P protein expression plasmid+L protein expression plasmid;

Treatment f): replicon launching plasmid+T7 DNA-dependent RNA polymerase expression plasmid+P protein expression plasmid+N protein expression plasmid;

Treatment g): replicon launching plasmid+T7 DNA-dependent RNA polymerase expression plasmid+L protein expression plasmid+N protein expression plasmid; and

Treatment h): replicon launching plasmid+T7 DNA-dependent RNA polymerase expression plasmid+L protein expression plasmid+N protein expression plasmid+P protein expression plasmid.

We observed glowing yeast cells in treatment groups g) and h) (indicating that GFP protein was expressed) but not in treatment groups a)-f).

### EXAMPLE 3

### MFSV Can Infect Insect Cells and Replicate in Them

Drosophila melanogaster Schneider (S)2 cells were incubated with 1 μg, 10 μg or 100 μg of MFSV and 50 μg/ml DEAE-dextran for 2 to 8 days post-inoculation. Using a two-step RT-PCR protocol that results in the amplification of an N protein mRNA fragment of about 1,100 bp but not the corresponding genomic RNA, we detected the transcript corresponding to the MFSV N gene.

We further conducted Western blot hybridizations with specific MFSV antibodies. Proteins of S2 cell extracts were separated by SDS-PAGE and transferred to nitrocellulose membranes, which were hybridized with MFSV antibodies. MFSV proteins were detected in S2 cells incubated with 10 μg and 100μ of MFSV and 50 μg/ml DEAE-dextran 2, 4, and 6 days post-inoculation. We compared the banding pattern of MFSV in S2 cells to those of MFSV-infected plants and MFSV virions and found them to be similar.

Immunofluorescence confocal microscopy was used to detect MFSV in S2 cells. S2 cells exposed to 100μ of MFSV and 50 μg/ml DEAE-dextran were fixed, stained with propidium iodide to detect nuclei (red fluorescence), and incubated with MFSV-specific primary antibodies and secondary goat anti-rabbit IgG labeled with Alexa Fluor 488 (green fluorescence). We detected MFSV in the S2 cells.

Although the invention has been described in connection with specific embodiments, it is understood that the invention is not limited to such specific embodiments but encompasses all such modifications and variations apparent to a skilled artisan that fall within the scope of the appended claims.

We claim:

1. An isolated nucleic acid comprising a polynucleotide or a complement of the polynucleotide wherein the polynucleotide encodes a polypeptide selected from SEQ ID NO:2, SEQ ID NO:3, SEQ ID NO:4, SEQ ID NO:5, SEQ ID NO:6, SEQ ID NO:7, and SEQ ID NO:8, with the proviso that the full length genomic RNA of maize fine streak virus (MFSV), the full length MFSV antigenomic RNA, and the corresponding DNA sequence of any of said two RNA molecules are excluded.

2. The isolated nucleic acid of claim 1, wherein the nucleic acid consists of a polynucleotide or a complement of the polynucleotide wherein the polynucleotide encodes a polypeptide selected from SEQ ID NO:2, SEQ ID NO:3, SEQ ID NO:4, SEQ ID NO:5, SEQ ID NO:6, SEQ ID NO:7, and SEQ ID NO:8.

3. A nucleic acid construct comprising the nucleic acid of claim 2 operable linked to a non-native promoter.

4. The nucleic acid construct of claim 3, wherein the nucleic acid construct is an expression vector.

5. A host cell comprising the nucleic acid construct of claim 3.

6. The host cell of claim 4, wherein the cell is a plant cell, an insect cell, or a yeast cell.

7. A DNA vector comprising a promoter and a terminator operably linked to a DNA construct wherein the DNA construct comprises an MFSV leader sequence, an MFSV trailer sequence, and between the leader and trailer sequences one or more DNA sequences of interest each of which is flanked at both ends by an MFSV gene junction sequence selected from SEQ ID NO:66-71, wherein a negative- or positive-strand RNA can be made from the DNA construct by a DNA-dependent RNA polymerase when the vector is provided in a suitable host cell, wherein the negative- or positive-strand RNA can be copied to each other in the suitable host cell when MFSV N protein, MFSV L protein, and MFSV P protein are provided in the cell, and wherein the negative-strand RNA can express the one or more DNA sequences of interest in the suitable host cell when MFSV N protein and MFSV L protein are provided in the cell.

8. The DNA vector of claim 7, wherein the DNA construct further comprises a ribozyme at one or both ends of the construct.

9. A kit comprising:
a first nucleic acid of claim 1, wherein the first nucleic acid comprises a first polynucleotide or its complement wherein the first polynucleotide encodes SEQ ID NO:2 (N protein);
a second nucleic acid of claim 1, wherein the second nucleic acid comprises a second polynucleotide or its complement wherein the second polynucleotide encodes SEQ ID NO:8 (L protein);
the vector of claim 7;
optionally, a third nucleic acid of claim 1, wherein the third nucleic acid comprises a third polynucleotide or its complement wherein the third polynucleotide encodes SEQ ID NO:3 (P protein); and
optionally, a fourth nucleic acid that comprises a fourth polynucleotide operably linked to a non-native promoter wherein the fourth polynucleotide encodes a DNA-dependent RNA polymerase.

10. An isolated polypeptide comprising an amino acid sequence selected from SEQ ID NO:2, SEQ ID NO:3, SEQ ID NO:4, SEQ ID NO:5, SEQ ID NO:6, SEQ ID NO:7, and SEQ ID NO:8.

11. The isolated polypeptide of claim 10, wherein the polypeptide consists of an amino acid sequence selected from SEQ ID NO:2, SEQ ID NO:3, SEQ ID NO:4, SEQ ID NO:5, SEQ ID NO:6, SEQ ID NO:7, and SEQ ID NO:8.

12. An antibody that specifically binds to the polypeptide of claim 11.

13. A method for expressing one or more DNA sequences of interest in a host cell, the method comprising the steps of:
providing a cell that is capable of producing a DNA-dependent RNA polymerase, an MFSV N protein as defined by SEQ ID NO:2, an MFSV L protein as defined by SEQ ID NO:8, and optionally an MFSV P protein as defined by SEQ ID NO:3;
introducing into the cell the DNA vector of claim 7; and
allowing the cell to produce the DNA-dependent RNA polymerase, the MFSV N protein, the MFSV L protein, and optionally the MFSV P protein so that the one or more DNA sequences of interest are expressed.

14. The method of claim 13, wherein the DNA construct in the DNA vector further comprises a ribozyme at one or both ends of the DNA construct.

15. The method of claim 13, wherein the cell is selected from a plant cell, an insect cell, and a yeast cell.

16. The method of claim 13, wherein the cell is a cell of a monocot plant.

17. The method of claim 13, wherein each of the N protein, the L protein, the P protein, and DNA-dependent RNA polymerase are produced by separate expression vectors in the cell.

18. The method of claim 13, wherein a negative-strand RNA is made from the DNA construct by the DNA-dependent RNA polymerase.

19. The method of claim 13, wherein the method is for expressing two or more DNA sequences of interest and the DNA construct comprises two or more DNA sequences of interest.

20. The method of claim 13, wherein the DNA construct comprises a DNA sequence that is complementary to the MFSV genomic RNA and MFSV viral particles are produced.

21. The method of claim 13, wherein the DNA construct comprises a DNA sequence that complementary to the MFSV antigenomic RNA and MFSV viral particles are produced.

22. The method of claim 13, wherein the DNA construct comprises a DNA sequence that is complementary to the MFSV genomic RNA or MFSV antigenomic RNA with one or more mutations and recombinant MFSV viral particles are produced.

23. The method of claim 13, wherein the method is for studying the effect of one or more MFSV genomic RNA mutations on viral particle production or infection efficiency by using a DNA construct that comprises a DNA sequence that is complementary to the MFSV genomic RNA or MFSV antigenomic RNA with one or more mutations and comparing viral production or infection efficiency to a control group that uses a DNA construct that comprises a DNA sequence that is complementary to the wild-type MFSV genomic RNA or wild-type MFSV antigenomic RNA.

24. A method for identifying a candidate agent that can potentially modulate the activity of MFSV, the method comprising the steps of:
providing a cell and introducing into the cell a DNA vector according to claim 13;
exposing the cell to a test agent; and
determining amount of wild-type or recombinant MFSV particles produced or the expression level of the DNA sequence of interest wherein a higher or lower amount of viral particles or a higher or lower level of expression of the DNA sequence of interest than that of a control cell not exposed to the test agent indicates that the test agent is a candidate for modulating the activity of MFSV.

25. The method of claim 24, wherein the method is for identifying a candidate agent that can potentially disrupt the activity of MFSV.
